(19) United States
(12) Patent Application Publication
(10) Pub. No.: US 2025/0352272 A1
(43) Pub. Date: Nov. 20, 2025

(54) REAL-TIME ULTRASOUND MONITORING FOR ABLATION THERAPY

(71) Applicant: The Johns Hopkins University, Baltimore, MD (US)

(72) Inventors: Emad M. BOCTOR, Ellicott City, MD (US); Chloe AUDIGIER, Baltimore, MD (US); Younsu KIM, Baltimore, MD (US); Emran ANAS, Baltimore, MD (US); Arman RAHMIM, Baltimore, MD (US); Nicholas P.K. ELLENS, Baltimore, MD (US); Xiaoyu GUO, Baltimore, MD (US)

(21) Appl. No.: 19/286,066

(22) Filed: Jul. 30, 2025

Related U.S. Application Data
(63) Continuation of application No. PCT/US2019/034330, filed on May 29, 2019.
(60) Provisional application No. 62/677,872, filed on May 30, 2018.

(51) Int. Cl. A61B 34/10; A61B 18/00; A61B 34/00; A61B 90/00

(52) U.S. Cl. CPC A61B 34/10; A61B 18/00; A61B 34/25; A61B 90/37; A61B 2018/00577; A61B 2018/00642; A61B 2018/00708; A61B 2018/00791; A61B 2034/104; A61B 2034/105; A61B 2090/378

(57) ABSTRACT

A system for monitoring an ablation therapy may include an ultrasound transmitter to transmit ultrasound signals through a region of tissue during an ablation procedure, an ultrasound receiver to receive the ultrasound signals after the ultrasound signals have passed through the region of tissue, and a signal processor to communicate with the ultrasound transmitter and the ultrasound receiver to obtain a set of measurements related to the ultrasound signals transmitted through the region of tissue during the ablation procedure. The signal processor may determine one or more acoustic characteristics of the ultrasound signals transmitted through the region of tissue based on the set of measurements and generate an image representing a thermal map of the region of tissue during the ablation procedure based on a mapping between the one or more acoustic characteristics of the ultrasound signals and changes in temperature.

### RELATED APPLICATION(S)

This application is a continuation of U.S. patent application Ser. No. 17/057,542, filed Nov. 20, 2020, which is a 371 national stage of PCTUS2019034330, filed May 29, 2019, entitled “REAL-TIME ULTRASOUND MONITORING FOR ABLATION THERAPY,” which claims priority to U.S. Provisional Application No. 62/677,872, filed May 30, 2018, the entire contents of each of which are hereby expressly incorporated herein by reference.

### GOVERNMENT LICENSE RIGHTS

This invention was made with government support under grant R01EB021396, awarded by the National Institutes of Health/NIH/DHHS; and grant IIS-0653322, awarded by the National Science Foundation. The government has certain rights in the invention.

### BACKGROUND

Tumor ablation therapy is an approach to remove tumor tissue by minimally invasive surgical procedures. In such procedures, an interventional ablation tool is typically directed to a target location within a body of a patient that is either close to, or within, the tumor tissue. Energy is then delivered to the tumor tissue in a sufficiently rapid manner to destroy the tumor tissue. The interventional ablation tool may be a radio frequency ablation tool, a laser ablation tool, a microwave ablation tool, a cryoablation tool, and/or the like. However, such interventional ablation tools are inaccurate and unsafe since the tools do not monitor temperature and/or a thermal dose.

### SUMMARY

According to some implementations, a system may include an ultrasound transmitter to transmit ultrasound signals through a region of tissue during an ablation procedure; an ultrasound receiver to receive the ultrasound signals transmitted by the ultrasound transmitter after the ultrasound signals pass through the region of tissue; and a signal processor communicatively coupled to the ultrasound transmitter and the ultrasound receiver. The signal processor may communicate with the ultrasound transmitter and the ultrasound receiver to obtain a set of measurements related to the ultrasound signals transmitted through the region of tissue during the ablation procedure, determine one or more acoustic characteristics of the ultrasound signals transmitted through the region of tissue based on the set of measurements, and generate an image representing a thermal map of the region of tissue during the ablation procedure based on a mapping between the one or more acoustic characteristics of the ultrasound signals and changes in temperature.

According to some implementations, a method may include obtaining patient-specific simulation data including expected temperature-dependent measurements for ultrasound signals to be transmitted through a region of tissue during an ablation procedure; determining a relative geometry between an ultrasound transmitter arranged to transmit the ultrasound signals through the region of tissue during the ablation procedure and an ultrasound receiver arranged to receive the ultrasound signals transmitted by the ultrasound transmitter after the ultrasound signals pass through the region of tissue; calculating actual temperature-dependent measurements for the ultrasound signals transmitted through the region of tissue during the ablation procedure based on the relative geometry between the ultrasound transmitter and the ultrasound receiver; and performing an action to guide the ablation procedure based on a comparison of the actual temperature-dependent measurements for the ultrasound signals and the expected temperature-dependent measurements for the ultrasound signals.

According to some implementations, a non-transitory computer-readable medium may store one or more instructions. The one or more instructions, when executed by one or more processors, may cause the one or more processors to determine relative locations associated with one or more ultrasound transmitters arranged to transmit ultrasound signals through a region of tissue during an ablation procedure and one or more ultrasound receivers arranged to receive the ultrasound signals transmitted by the one or more ultrasound transmitters after the ultrasound signals pass through the region of tissue. The one or more instructions, when executed by the one or more processors, may cause the one or more processors to calculate a set of temperature-dependent measurements for the ultrasound signals transmitted through the region of tissue during the ablation procedure and determine, based on the set of temperature-dependent measurements and the relative locations associated with the one or more ultrasound transmitters and the one or more ultrasound receivers, one or more acoustic characteristics of the ultrasound signals transmitted through the region of tissue, wherein the one or more acoustic characteristics include one or more of a speed, an intensity, an attenuation, a phase, or a nonlinearity for the ultrasound signals. The one or more instructions, when executed by the one or more processors, may cause the one or more processors to generate an image representing a thermal map of the region of tissue during the ablation procedure based on temperature-dependent variations in the one or more acoustic characteristics of the ultrasound signals.

### BRIEF DESCRIPTION OF THE DRAWINGS

FIGS. 1-4 are diagrams of example interventional systems with real-time ablation thermal dose monitoring.

FIG. 5 is a diagram of example ablation tool designs for ultrasound thermal monitoring.

FIG. 6 is a diagram of an example ultrasound element control system.

FIG. 7 is a diagram of example components of one or more devices of FIGS. 1-6.

FIG. 8A is a diagram of example results of a method for mapping changes in acoustic properties during thermal ablation to temperature changes.

FIG. 8B is a diagram of an example method for reconstructing an image representing a thermal map in a region of tissue underdoing thermal ablation.

FIG. 8C is a diagram of an example intersecting length distribution through each layer at different sensor locations.

FIG. 9A is a diagram depicting that intra-operative data and thermal simulations can be combined to improve real-time monitoring of thermal ablation.

FIG. 9B is a diagram depicting how intra-operative data can be regarded as an input of a thermal computational simulation to allow model parameter estimation and provide improved real-time thermal monitoring.

FIG. 9C is a diagram of an example model to obtain improved temperature monitoring during thermal ablation.

FIG. 9D is a diagram depicting how a thermal propagation simulation can be used to improve thermal map reconstruction from intra-operative ultrasound measurements as pattern injection or ultrasound time of flight acquisition.

FIG. 9E is a diagram of an example patient-specific simulation method.

FIG. 9F is a diagram depicting a predictive power of a radiofrequency ablation model.

FIG. 9G is a diagram depicting how a framework can be implemented so that a deep learning engine may enable HIFU guidance.

FIG. 9H is a diagram of an example framework for simulation-driven monitoring of HIFU ablation.

FIG. 9I is a diagram of an example method implemented and tested on phantom data.

FIG. 10A is a diagram of an example experiment that has been performed to illustrate reconstruction of temperature changes by analyzing injected pattern deformation.

FIG. 10B is a diagram depicting an example method for recovering a thermal map based on simulated pattern injection using a biophysical thermal ablation model.

FIG. 11A is a diagram of an example configuration of standing wave elastography.

FIG. 11B is a diagram of an example experimental result of a plastisol phantom.

FIG. 11C is a diagram of an example relaxation process of a gelatin phantom in an ultrasound standing wave off period.

FIG. 12A is a diagram of an example architecture of a deep neural network for prediction of thermal maps from a set of given radio frequency (RF) signals.

FIG. 12B is a diagram of example architectures of convolutional neural network high-pass filtering (CNNHPF) and CNN image de-noising (CNNIDN).

FIG. 12C is a diagram of an example architecture of a deep neural network for predicting thermal maps from a given set of images.

FIGS. 13-15 are flow charts of example processes for real-time ultrasound monitoring for ablation therapy.

### DETAILED DESCRIPTION

The following detailed description of example implementations refers to the accompanying drawings. The same reference numbers in different drawings may identify the same or similar elements.

During a heat-induced tumor ablation treatment, a cryoablation treatment in which extreme cold is used to destroy targeted tissue, and/or the like, guiding and monitoring the ablation process is crucial, especially when the operation requires high accuracy. However, due to a low contrast between ablated and untreated tissue in ultrasound images (e.g., B-mode images), conventional ultrasound imaging is usually not effective for the monitoring. Other imaging modalities, including computed tomography (CT) and magnetic resonance imaging (MRI), can be incorporated with the ablation therapy and provide effective image guidance and monitoring. However, these high-end imaging devices have requirements that may make this approach unaffordable or inaccessible for many patients. The radiation dose and magnetic field compatibility requirements also prevent these methods from being widely used.

Some implementations described herein provide real-time ultrasound temperature monitoring systems for ablation therapy using a high-intensity focused ultrasound (HIFU) system. For example, some implementations described herein may utilize one or more of several different thermal dose monitoring systems and methods, which may be based on ultrasound imaging modalities, to assist an operator with control of an ablation treatment process. One or more of these methods can be implemented in an interventional ablation system to provide a thermal dose at a low cost and with zero radiation. Furthermore, these methods enable real-time, high-accuracy guidance and monitoring during ablation therapy, thus reducing the risk and difficulty of the ablation treatment. Furthermore, these methods can be used to monitor and/or guide any suitable ablation treatment modality, including radiofrequency ablation, laser ablation, microwave ablation, cryoablation, and/or the like.

In this way, some implementations described herein provide a new configuration of an ultrasound imaging system that includes an ablation applicator with ultrasound transceiver sources (e.g., transducer elements formed from a piezoelectric (PZT) material, a polyvinylidene difluoride (PVDF) material, and/or the like) that can generate imaging pulses using a photoacoustic effect, and an external transducer array arranged to receive a signal. Some implementations described herein may provide acoustic radiation force imaging (ARFI) in an intra-ablation region, where acoustic radiation force (ARF) pulses may be generated by a transducer attached to the ablation applicator, and an image representing a thermal map of the intra-ablation region may be generated in real-time using a synchronized ultrasound imaging array. Some implementations described herein may also provide imaging reconstruction models that can be used to generate the image representing the thermal map of the intra-ablation region.

The terms “light” and “optical” are intended to have a broad meaning. These terms can include visible regions of the electromagnetic spectrum. Additionally, or alternatively, these terms can include nonvisible regions of the electromagnetic spectrum such as infrared light, ultraviolet light, and/or the like.

The term “photoacoustic” is intended to have a broad meaning, which can include photons at any energy suitable for the particular application in which energy that generates an acoustic signal is deposited in a body of interest.

The term “body” is intended to refer generally to a mass, and not necessarily or specifically to a human or animal body. In some implementations, a body of interest can include a human or animal organ, or a portion thereof.

The term “interstitial” means to be inserted into tissue, such as a needle inserted into tissue with the inserted tip being surrounded by the tissue.

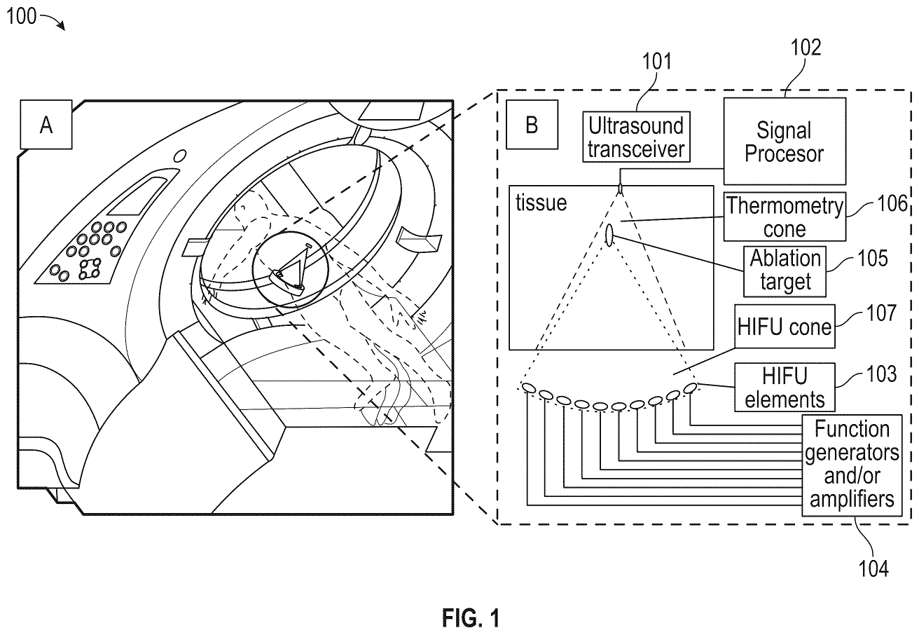

FIG. 1 is a diagram of an example interventional system 100 with real-time ablation thermal dose monitoring. In some implementations, FIG. 1 depicts ultrasound thermal dose monitoring of a magnetic resonance (MR)-compatible HIFU system. Portion (A) of FIG. 1 depicts a three-dimensional rendering model that shows the HIFU system embedded in a patient bed, external MR-compatible ultrasound receivers, and an MRI gantry. Portion (B) of FIG. 1 depicts a zoomed-in schematic diagram that highlights individually controlled HIFU elements with function generators and/or amplifiers 104, a set of HIFU elements 103 (e.g., MR-compatible ultrasound sensors, transducer elements, and/or the like), a HIFU cone 107 (e.g., a cone-shaped profile through which transmitted ultrasound signals propagate), and an ultrasound (US) thermometry cone 106. For example, ultrasound signals transmitted using the HIFU elements 103 may pass through a region of tissue that includes an ablation target 105 (e.g., tumor tissue, necrotic tissue, uterine fibroids, and/or other soft tissue targeted by an ablation procedure). The ultrasound signals may propagate through the HIFU cone 107, and the thermometry cone 106 may focus energy of the ultrasound signals around the ablation target 105 to destroy the ablation target 105 during an ablation procedure.

In some implementations, as shown in FIG. 1, the interventional system 100 may include one or more ultrasound transceivers 101, a signal processor 102, HIFU elements 103, and function generators and/or amplifiers 104. In some implementations, the signal processor 102 may communicate with the ultrasound transceivers 101 and/or the HIFU elements 103 to acquire time of flight and intensity information to calculate a thermal map of the region of tissue.

In some implementations, the ultrasound transceivers 101 and/or HIFU elements 103 may include one or more piezoelectric transducers, one or more photoacoustic transmitters and/or receivers, and/or the like. Furthermore, in some implementations, the ultrasound transceivers 101 and/or HIFU elements 103 may include one or more ultrasound transmitters and/or receivers described in U.S. Patent Application Publication No. 2014/0024928, the content of which is incorporated herein by reference in its entirety.

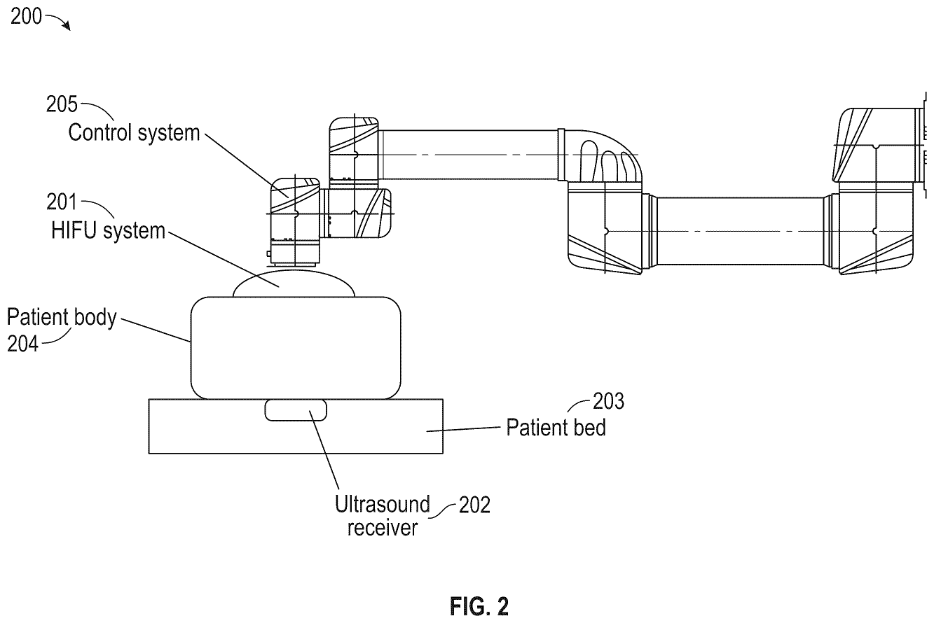

FIG. 2 is a diagram of an example interventional system 200 with real-time ablation thermal dose monitoring. In some implementations, the interventional system 200 depicted in FIG. 2 may include a control system 205 (e.g., one or more robot arms, motors, actuators, linear stages, and/or the like) holding a HIFU system 201. Furthermore, as shown in FIG. 2, the interventional system 200 may include an ultrasound receiver 202 and a patient bed 203. In some implementations, movement and location of the HIFU system 201 may be controlled by the control system 205, and focused ultrasound signals may be transmitted through a region of tissue in a patient body 204 positioned in patient bed 203 to eliminate targeted soft tissue in the patient body 204. In some implementations, one or more elements of HIFU system 201 may communicate with one or more elements of an ultrasound transceiver (e.g., ultrasound receiver 202) to monitor a thermal dose delivered to the patient body 204 during the ablation procedure.

In some implementations, ultrasound data used to monitor the thermal dose can be collected sequentially for a non-invasive ablation procedure. For example, the HIFU system 201 may transmit ultrasound pulses and a trigger signal, and the trigger signal may initiate collection of ultrasound data received by one or more external elements. By example interventional system 200 repeating this procedure, ultrasound pulses transmitted from each element in the HIFU system 201 may be collected.

In some implementations, the ultrasound data used to monitor the thermal dose can be collected sequentially for a minimally-invasive ablation procedure. For example, each element in an ultrasound transducer may transmit ultrasound pulses and a trigger signal, and the trigger signal may initiate collection of ultrasound data received by one or more external elements. By example interventional system 200 repeating this procedure, ultrasound pulses transmitted from each ultrasound element in the ultrasound transducer may be collected.

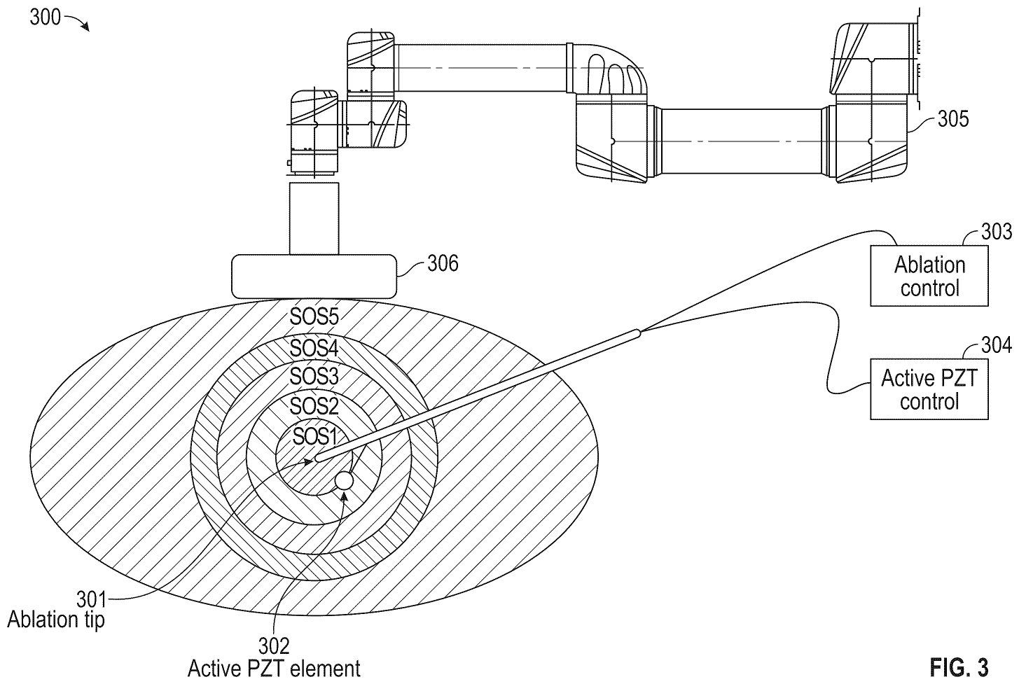

FIG. 3 is a diagram of an example interventional system 300 with real-time ablation thermal dose monitoring. In some implementations, interventional system 300 may include an ablation device 301 (e.g., a catheter that may be inserted into targeted tissue and used to transmit ultrasound pulses or signals at different locations), one or more ultrasound transducers 302 (e.g., active PZT elements), an ablation control system 303, an active PZT control system 304, a control system 305 (e.g., one or more robot arms, motors, actuators, linear stages, and/or the like), and an ultrasound probe 306. In some implementations, the ultrasound transducers 302 may be used to transmit ultrasound signals, and the ultrasound probe 306 may receive the ultrasound signals. In some implementations, the ultrasound probe 306 may acquire time of flight data in a path between the ultrasound signals. In some implementations, the one or more ultrasound transducers 302 may receive or otherwise detect ultrasound signals that are transmitted from ultrasound probe 306. In such implementations, the active PZT control system 304 may receive a trigger signal from the ultrasound probe 306.

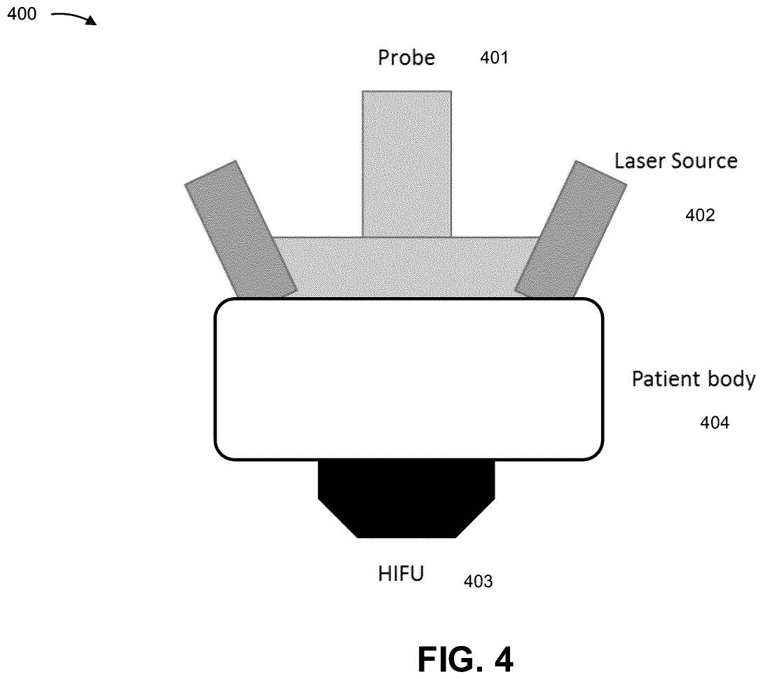

FIG. 4 is a diagram of an example interventional system 400 with real-time ablation thermal dose monitoring. In some implementations, FIG. 4 depicts an example alignment among photoacoustic, HIFU, and ultrasound receiver elements. For example, as shown in FIG. 4, one or more photoacoustic laser sources 402 may be engaged with an ultrasound probe 401, and the ultrasound probe 401 may acquire a photoacoustic signal and ultrasound signals from a HIFU system 403. By utilizing the signals from the HIFU system 403 and photoacoustic ultrasound, the interventional system 400 may be used to monitor thermal changes in a patient body 404 during an ablation procedure.

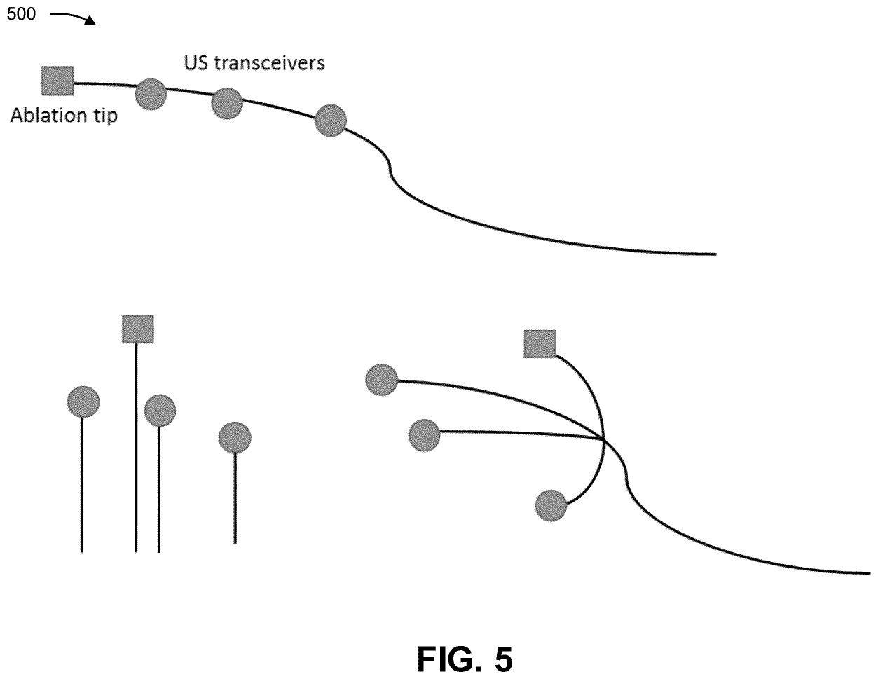

FIG. 5 is a diagram of examples of ablation tool designs 500 that can be used for ultrasound thermal monitoring in interventional systems 100, 200, 300, 400, and/or the like. As shown in FIG. 5, each of the ablation tool designs 500 may include an ablation tip and one or more ultrasound transceivers, which may be arranged in a variety of configurations.

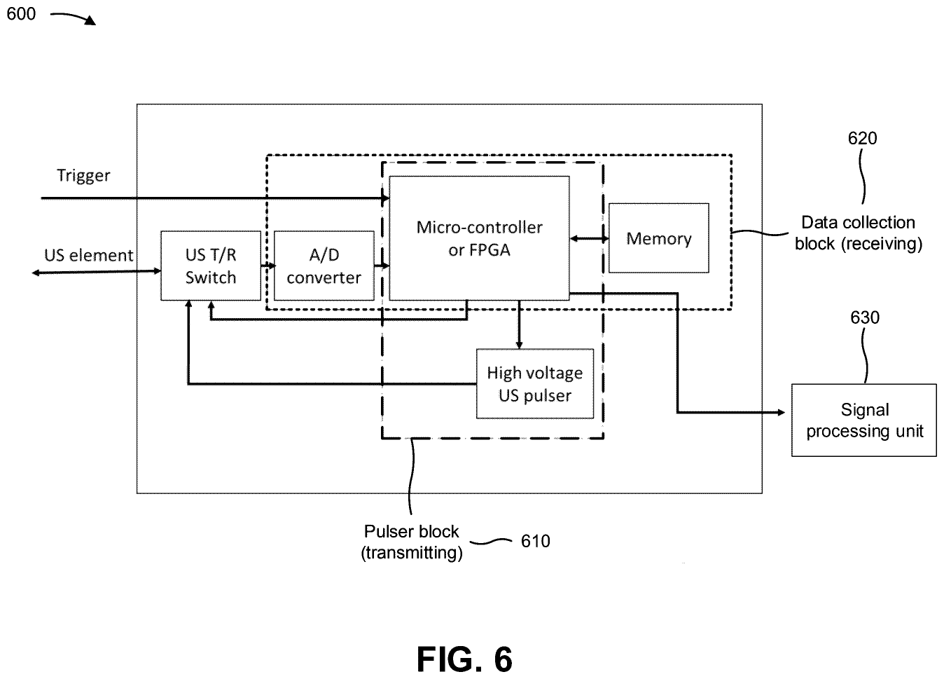

FIG. 6 is a diagram of an example ultrasound element control system 600. As shown, ultrasound element control system 600 may include an ultrasound transmit/receive switch (US T/R Switch), an analog-to-digital (A/D) converter, a micro-controller or field-programmable gate array (FPGA), a memory, and a high-voltage ultrasound pulser. In some implementations, the micro-controller and the high-voltage ultrasound pulser may represent a pulser (e.g., transmitting) block 610, and the A/D converter, the micro-controller, and the memory may represent a data collection (e.g., receiving) block. In some implementations, the ultrasound element control system 600 may be coupled to a signal processing unit 630, which may include and/or correspond to a computing device, a display device, and/or the like.

As indicated above, FIGS. 1-6 are provided merely as one or more examples. Other examples may differ from what is described with regard to FIGS. 1-6. For example, although FIG. 1-6 are described in a context of HIFU systems that use ultrasound waves to heat targeted tissue to be destroyed, in various implementations, an ablation treatment may be performed using any suitable technique such as radiofrequency ablation, laser ablation, microwave ablation, cryoablation, and/or the like.

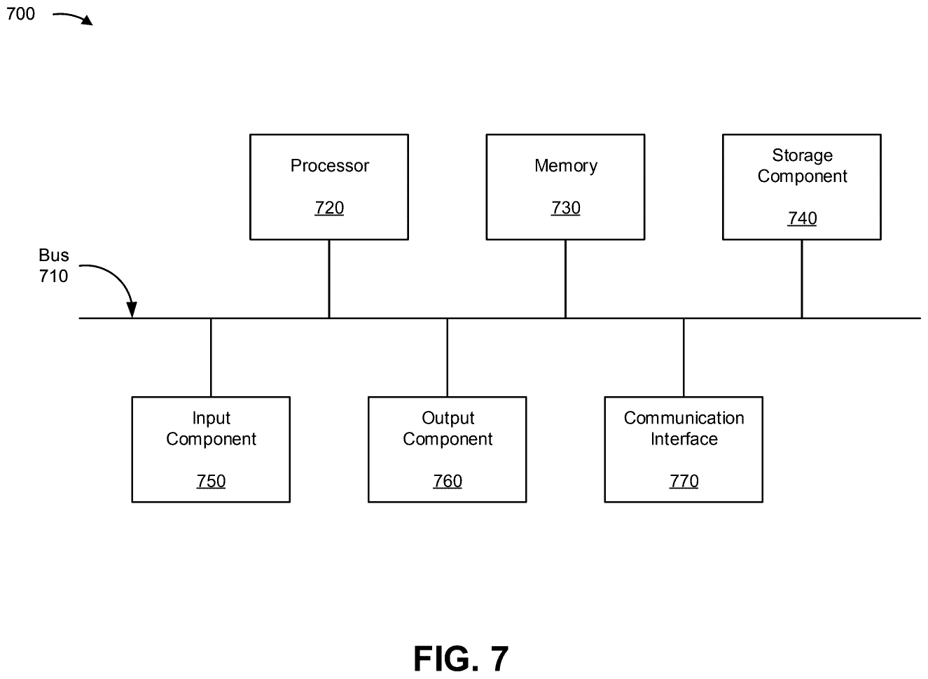

FIG. 7 is a diagram of example components of a device 700. Device 700 may correspond to one or more devices depicted in FIGS. 1-6. For example, device 700 may correspond to ultrasound transceiver 101, signal processor 102, HIFU element 103, function generator and/or amplifier 104, HIFU system 201 and/or 403, ultrasound receiver 202, control system 205 and/or 305, ablation device 301, ultrasound transducer 302, ablation control system 303, active PZT control system 304, ultrasound probe 306, 401, ultrasound element control system 600, pulser block 610, data collection block 620, signal processing unit 630, and/or the like. In some implementations, ultrasound transceiver 101, signal processor 102, HIFU element 103, function generator and/or amplifier 104, HIFU system 201 and/or 403, ultrasound receiver 202, control system 205 and/or 305, ablation device 301, ultrasound transducer 302, ablation control system 303, active PZT control system 304, ultrasound probe 306, 401, ultrasound element control system 600, pulser block 610, data collection block 620, and/or signal processing unit 630 may include one or more devices 700 and/or one or more components of device 700. As shown in FIG. 7, device 700 may include a bus 710, a processor 720, a memory 730, a storage component 740, an input component 750, an output component 760, and a communication interface 770.

Bus 710 includes a component that permits communication among multiple components of device 700. Processor 720 is implemented in hardware, firmware, and/or a combination of hardware and software. Processor 720 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 720 includes one or more processors capable of being programmed to perform a function. Memory 730 includes a random-access memory (RAM), a read only memory (ROM), and/or another type of dynamic or static storage device (e.g., a flash memory, a magnetic memory, and/or an optical memory) that stores information and/or instructions for use by processor 720.

Storage component 740 stores information and/or software related to the operation and use of device 700. For example, storage component 740 may include a hard disk (e.g., a magnetic disk, an optical disk, and/or a magneto-optic disk), a solid-state drive (SSD), a compact disc (CD), a digital versatile disc (DVD), a floppy disk, a cartridge, a magnetic tape, and/or another type of non-transitory computer-readable medium, along with a corresponding drive.

Input component 750 includes a component that permits device 700 to receive information, such as via user input (e.g., a touch screen display, a keyboard, a keypad, a mouse, a button, a switch, and/or a microphone). Additionally, or alternatively, input component 750 may include a component for determining location (e.g., a global positioning system (GPS) component) and/or a sensor (e.g., an accelerometer, a gyroscope, an actuator, another type of positional or environmental sensor, and/or the like). Output component 760 includes a component that provides output information from device 700 (via, e.g., a display, a speaker, a haptic feedback component, an audio or visual indicator, and/or the like).

Communication interface 770 includes a transceiver-like component (e.g., a transceiver, a separate receiver, a separate transmitter, and/or the like) that enables device 700 to communicate with other devices, such as via a wired connection, a wireless connection, or a combination of wired and wireless connections. Communication interface 770 may permit device 700 to receive information from another device and/or provide information to another device. For example, communication interface 770 may include an Ethernet interface, an optical interface, a coaxial interface, an infrared interface, a radio frequency (RF) interface, a universal serial bus (USB) interface, a wireless local area network interface, a cellular network interface, and/or the like.

Device 700 may perform one or more processes described herein. Device 700 may perform these processes based on processor 720 executing software instructions stored by a non-transitory computer-readable medium, such as memory 730 and/or storage component 740. As used herein, the term “computer-readable medium” refers to a non-transitory memory device. A memory device includes memory space within a single physical storage device or memory space spread across multiple physical storage devices.

Software instructions may be read into memory 730 and/or storage component 740 from another computer-readable medium or from another device via communication interface 770. When executed, software instructions stored in memory 730 and/or storage component 740 may cause processor 720 to perform one or more processes described herein. Additionally, or alternatively, hardware circuitry may be used in place of or in combination with software instructions to perform one or more processes described herein. Thus, implementations described herein are not limited to any specific combination of hardware circuitry and software.

The number and arrangement of components shown in FIG. 7 are provided as an example. In practice, device 700 may include additional components, fewer components, different components, or differently arranged components than those shown in FIG. 7. Additionally, or alternatively, a set of components (e.g., one or more components) of device 700 may perform one or more functions described as being performed by another set of components of device 700.

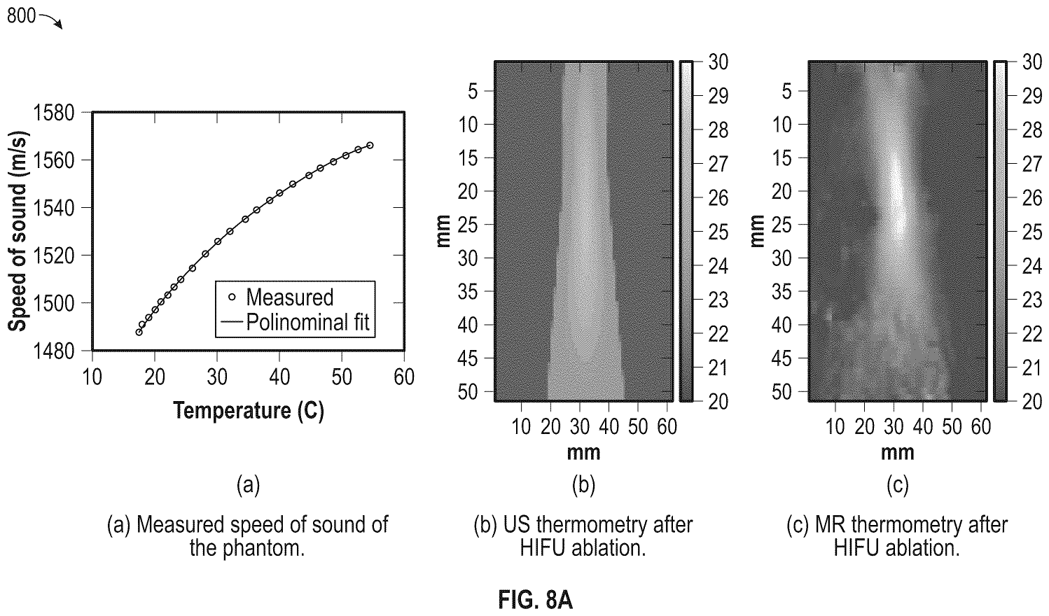

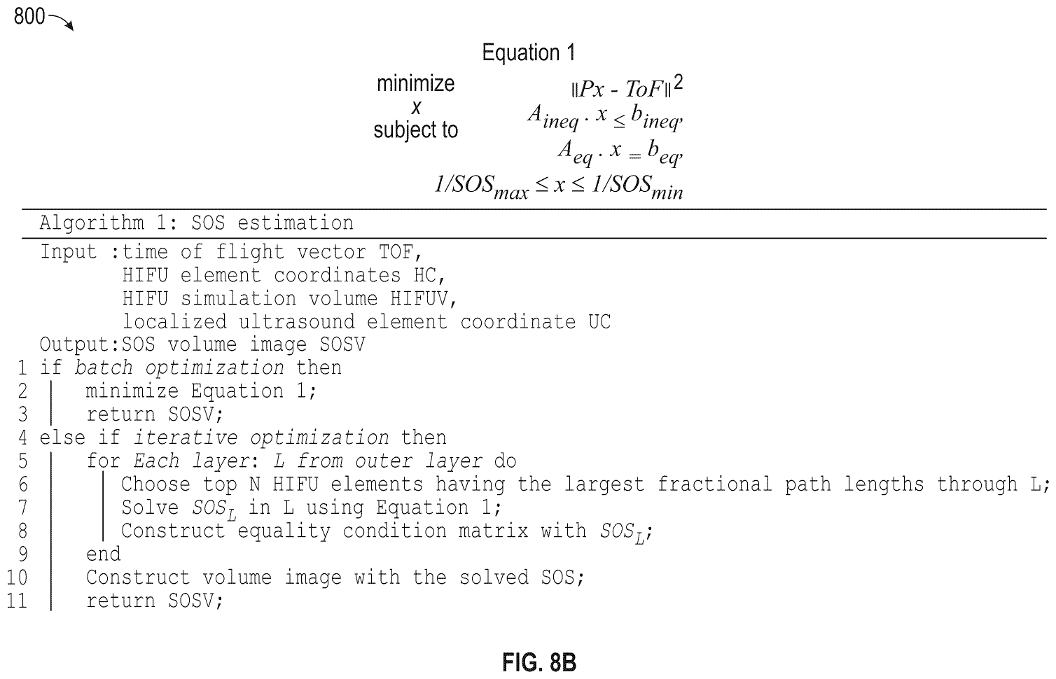

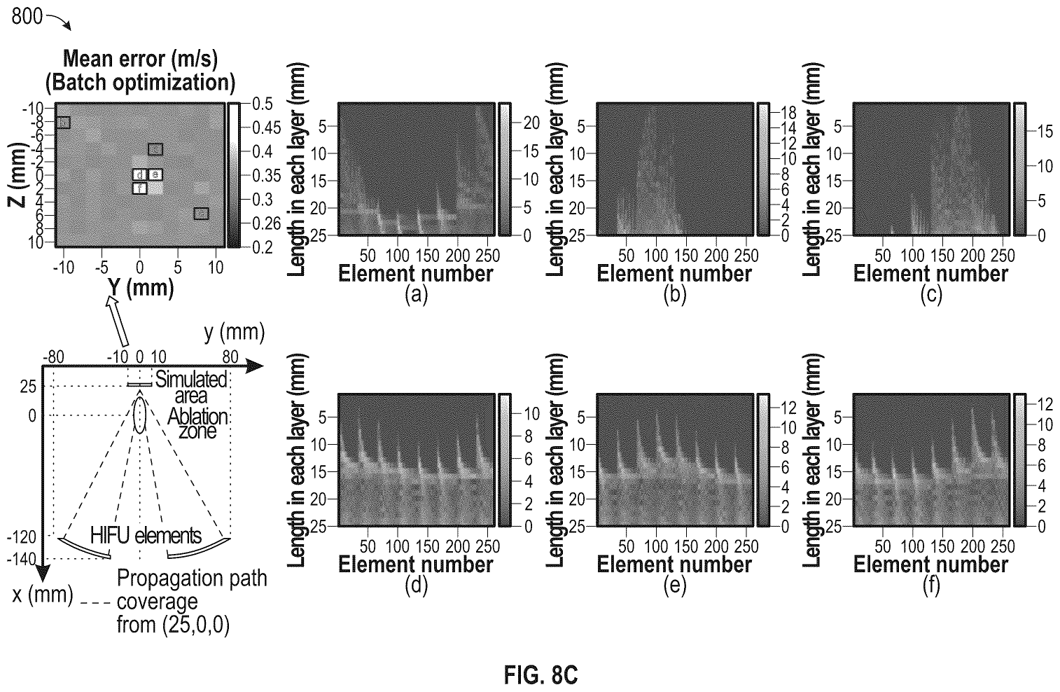

In some implementations, FIGS. 8A-8C depict a first example method 800 that may be implemented in one or more systems and/or devices described elsewhere herein (e.g., systems and/or devices described above in connection with FIGS. 1-7). The first example method 800 may include mapping changes in acoustic properties during an ablation treatment to changes in temperature. For example, in some implementations, the ablation treatment may be described herein in a context that relates to a HIFU ablation treatment. However, the processes and techniques described herein to monitor and/or guide the HIFU ablation treatment may be applied to monitor and/or guide a cryoablation treatment, a laser ablation treatment, and/or the like.

In the first example method 800, with reference to the example interventional system 100 shown in FIG. 1, changes in acoustic properties may be mapped to changes in temperature during the ablation treatment by configuring the signal processor 102 to calculate a temperature delivered to the region of tissue that includes the ablation target 105 in real-time based on time of flight (ToF) measurements associated with ultrasound signals that are transmitted by HIFU elements 103 to external ultrasound transceivers 101. For example, as mentioned elsewhere herein, the ultrasound signals may be transmitted through the region of tissue undergoing the ablation procedure and regions immediately surrounding the region undergoing the ablation procedure.

The method 800 for mapping changes in acoustic properties to changes in temperature may be based on various ultrasound characteristics that are temperature-dependent, which may include changes in the speed of sound (SoS) at different temperatures. Furthermore, in some implementations, the temperature-dependent ultrasound characteristics may include attenuation, phase, nonlinearity, and/or other characteristics that relate to an intensity of an ultrasound signal (e.g., a concentration of energy in a beam) at different temperatures. Accordingly, to map the changes in acoustic properties to the changes in temperature, the ultrasound transceivers 101 may communicate with the HIFU elements 103 to acquire time of flight measurements and other acoustic properties in order to calculate thermal maps.

For image reconstruction (e.g., generating an image that represents the thermal map), a distance between the ultrasound transceivers 101 and the HIFU elements 103 may be determined in various ways. For example, the distance may be determined based on imaging, using an optical tracker to locate the ultrasound transceivers 101 and/or HIFU elements 103, using encoders to track change in the position of the ultrasound transceivers 101 and/or HIFU elements 103, using ultrasound triangulation localization, using a fixed system (e.g., a robotic arm as shown in FIG. 2 and/or FIG. 3) that has a transmitter and a receiver at fixed distances from each other, and/or the like. The distance between the ultrasound transceivers 101 and the HIFU elements 103 may ensure that MRI images provide a relative geometry between external elements and coordinates of a focused ultrasound (FUS) system.

In some implementations, one or more tomographic methods may be utilized to map a temperature in a volume and to derive unknown parameters. For example, in some implementations, the tomographic methods may perform imaging by sections or sectioning to produce a three-dimensional image of internal structures of a solid object through the use of penetrating waves. One example tomographic method may include utilizing multiple ultrasound transceivers 101 that receive ultrasound pulses from different known locations. As a result, a quantity of independent equations may be greater than a quantity of unknowns, and image reconstruction becomes a solvable problem.

Additionally, or alternatively, a quantity of FUS elements generating pulses to an external receiver may be optimized to limit pulse generation to effective FUS elements that transmit ultrasound signals that travel through an ablation zone and carry valuable ToF information. In some implementations, several MRI-compatible receivers may be utilized, and real-time MRI thermometry may be utilized as a reference to assess and optimize system configurations and thermal image generation methods.

In some implementations, by sweeping a patient body surface with ultrasound transceivers (e.g., using a robot arm associated with a control system, as shown in FIG. 2 and/or FIG. 3), ultrasound data can be acquired via two-dimensional ultrasound sensor arrays. Ultrasound receivers may include ultrasound sensors, ultrasound probes, two-dimensional pressure sensors, and/or other sensors that can detect ultrasound signals transmitted by one or more HIFU elements, ultrasound transmitters, and/or the like. In some implementations, the ultrasound receivers may include a conventional array, a series of single receivers on a frame with known dimensions, and/or the like. In some implementations, a catheter may be moved inside the tissue and used to transmit the ultrasound pulses at different locations (e.g., as shown in FIG. 3).

In some implementations, a region of interest may be reduced to an area where a temperature is actually changing. As described in further detail elsewhere herein, such implementations may use a thermal propagation model to segment a region, and voxels that include similar temperatures may be grouped together and may decrease a total number of unknowns.

With reference to FIG. 8A, because the initial speed of sound and a structure of the HIFU elements 103 may be known, locations of the ultrasound transceivers 101 with respect to the HIFU elements 103 can be identified from time of flight (ToF) data acquired before the ablation procedure. Accordingly, changes in the ToF data may be analyzed to create thermal maps in real-time during the ablation procedure. The left side (part (a)) of FIG. 8A depicts an evolution of the speed of sound with respect to temperature (e.g., as measured for a phantom), and the right side (parts (b) and (c)) of FIG. 8A depicts a reconstructed thermal map after HIFU ablation using the ultrasound thermometry method described herein (e.g., mapping changes in acoustic characteristics of ultrasound signals to changes in temperature), and a magnetic resonance (MR) thermal map acquired after the same HIFU ablation. As shown in FIG. 8A, the reconstructed thermal map using the ultrasound thermometry method described herein accurately corresponds to the MR thermal map without requiring the use of MRI equipment that may be prohibitively expensive.

In some implementations, various reconstruction methods can be used to generate speed-of-sound (SoS) volumes that can in turn be used to generate the thermal maps. For example, an SoS volume may be generated using a batch optimization technique in which the SoS volume is generated using all available ToF information, an iterative optimization technique in which the SoS volume is generated for various segmented regions, and/or the like. For example, FIG. 8B illustrates a diagram of an example reconstruction method for SoS estimation, in which a time of flight vector, HIFU element coordinates, a HIFU simulation volume, and localized ultrasound element coordinates are provided as inputs and an SoS volume image is produced as an output. For example, when the batch optimization technique is used to produce the SoS volume image, the batch optimization technique may be configured to minimize an expression containing one or more parameters subject to one or more constraints, as shown on the left-hand side of FIG. 8B. Additionally, or alternatively, when the iterative optimization technique is used to produce the SoS volume image, a given quantity of HIFU elements in a particular layer that have largest fractional path lengths through the layer may be chosen, and the equation shown on the left-hand side of FIG. 8B may be used to solve the SoS volume in the particular layer. An equality condition matrix may then be constructed with the SoS volume in the particular layer, and this process may be repeated for each layer. Accordingly, the SoS volume image can then be constructed based on the SoS volume in each layer. In some implementations, the reconstruction method used to solve for the SoS volume can further include analyzing an intersection length distribution at different sensor locations. For example, FIG. 8C illustrates example intersecting length distributions through each layer at different sensor locations.

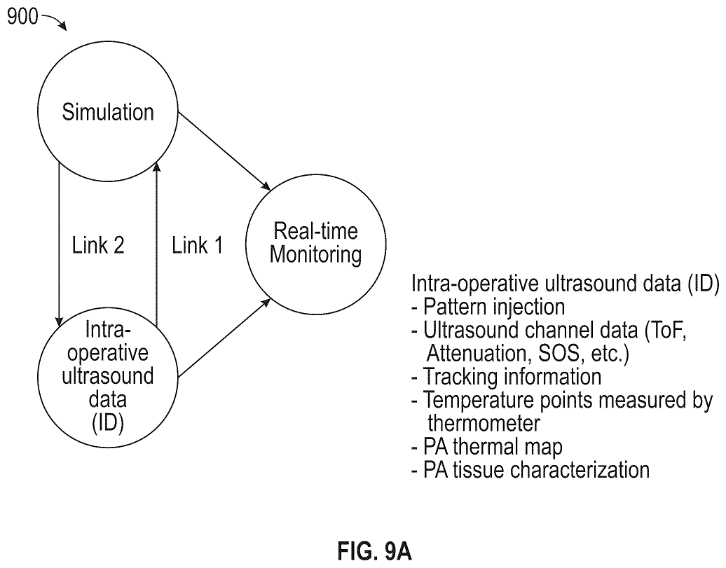

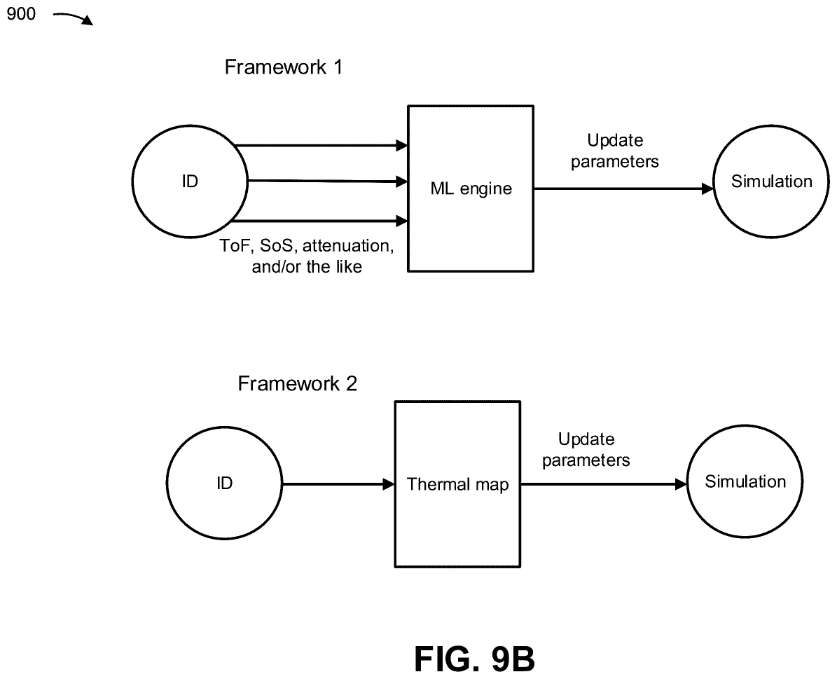

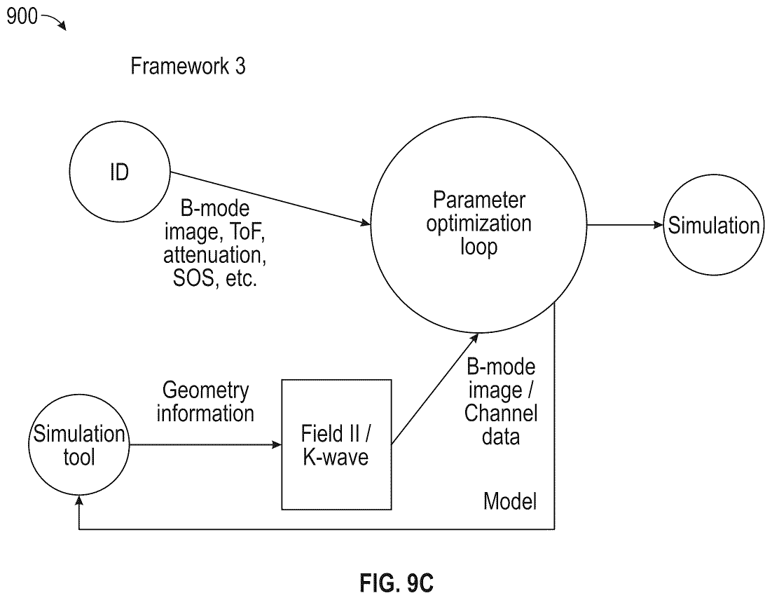

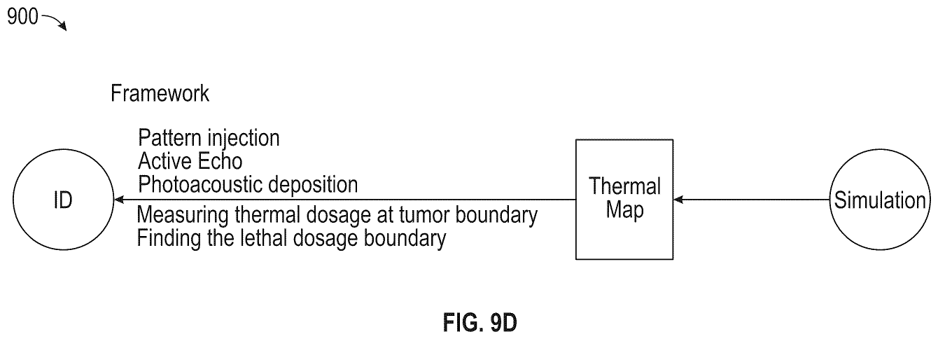

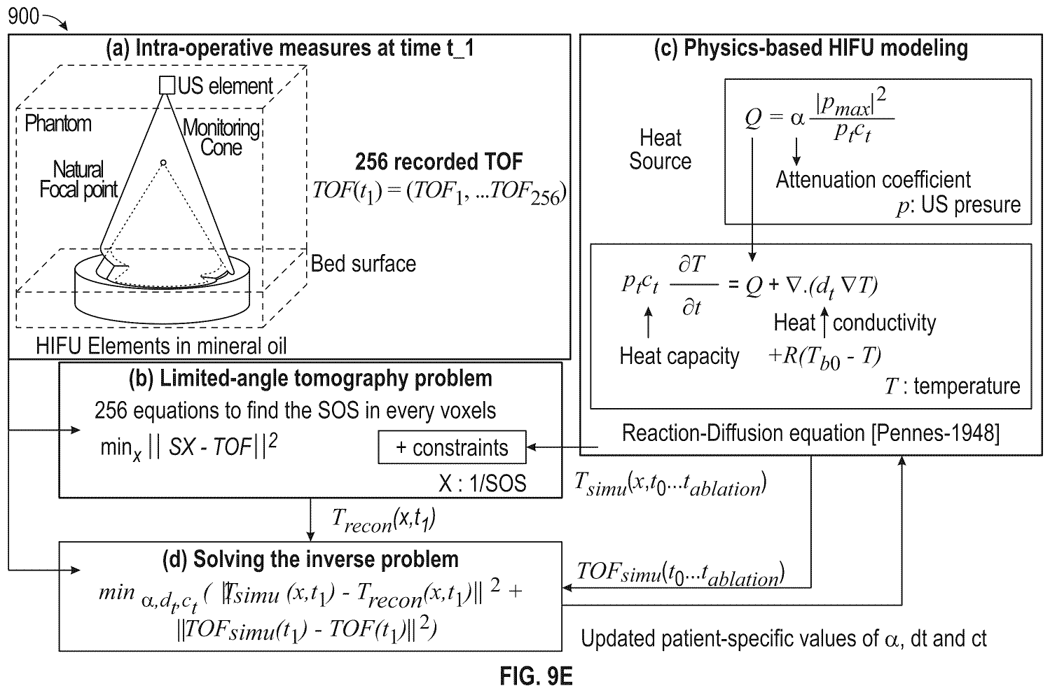

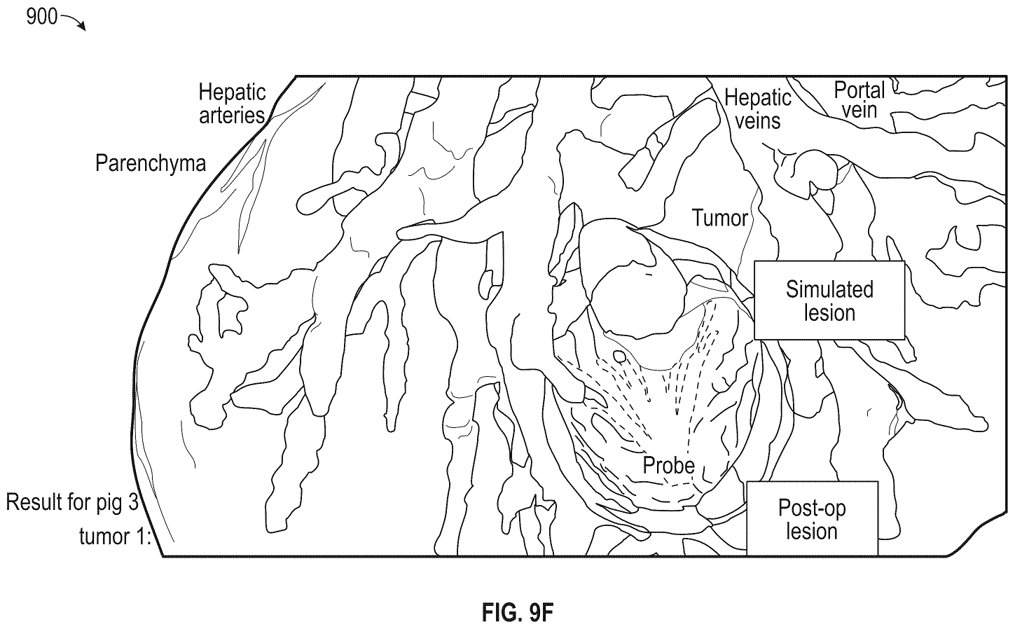

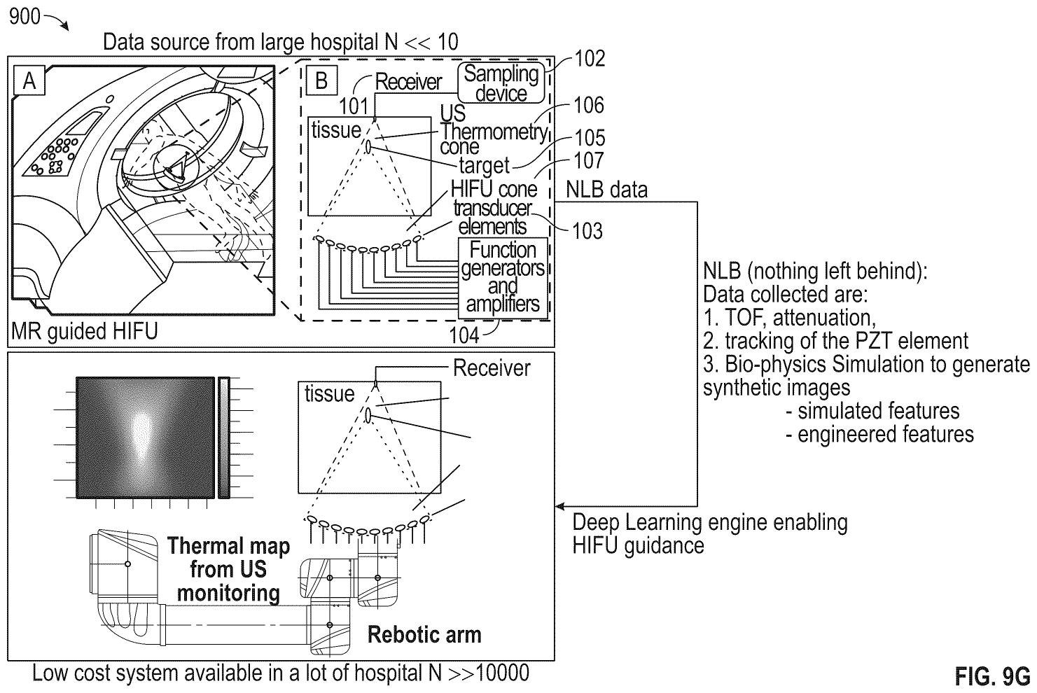

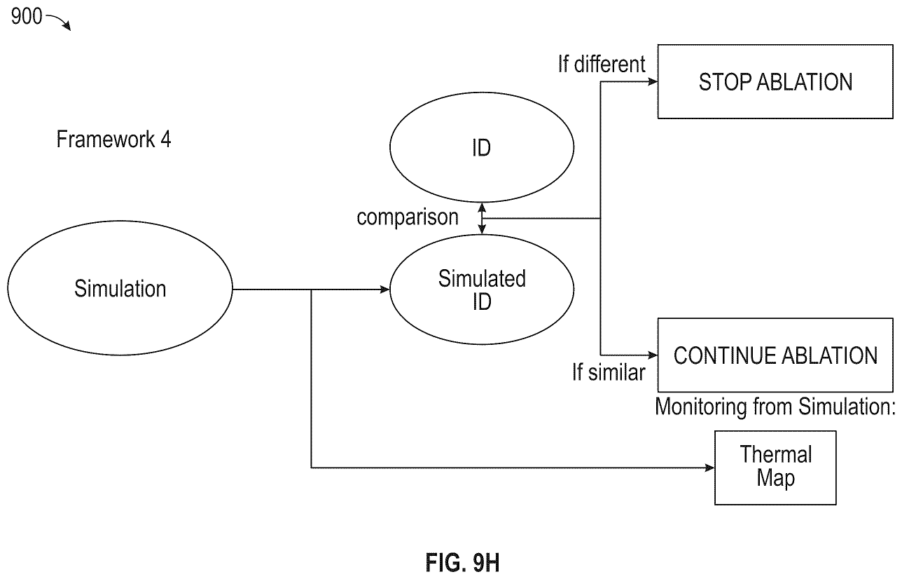

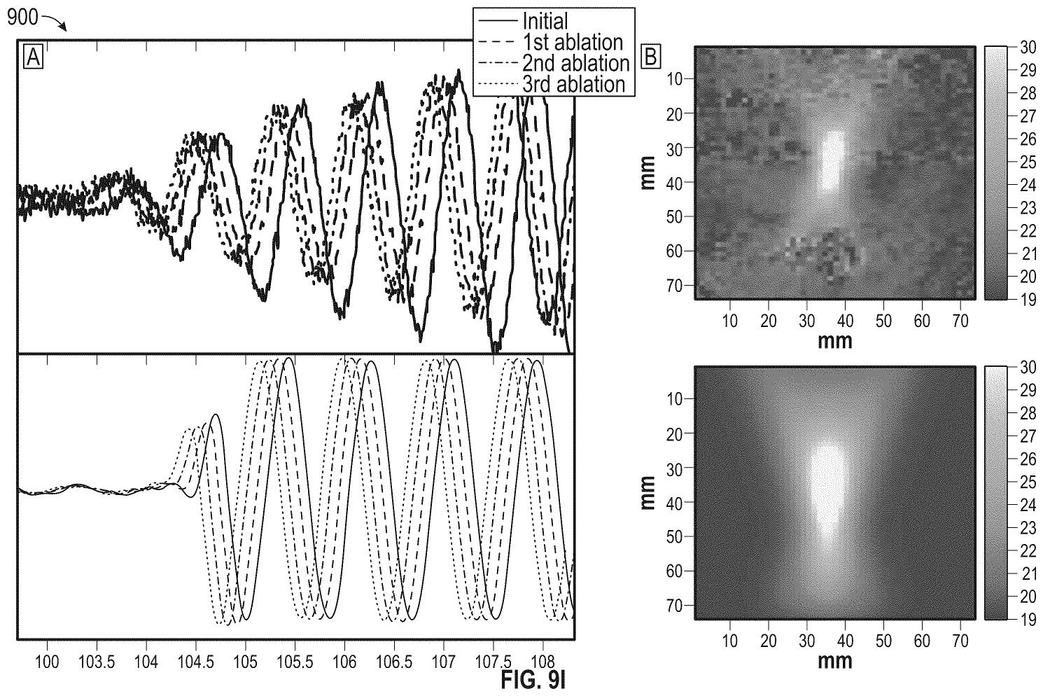

In some implementations, FIGS. 9A-9I depict a second example method 900 that may be implemented in one or more systems and/or devices described elsewhere herein (e.g., systems and/or devices described above in connection with FIGS. 1-7). The second example method 900 may include simulation coupled with reconstruction. For example, FIG. 9A is a diagram depicting that intra-operative data and thermal simulations can be combined to improve real-time monitoring of thermal ablation. For example, the intra-operative data may include pattern injection data, ultrasound channel data (e.g., time of flight, attenuation, speed of sound, and/or the like), tracking information, temperature points (e.g., as measured using a thermometer), photoacoustic (PA) thermal map data, PA tissue characterization data, and/or the like. In some implementations, a thermal propagation model may provide temperature and thermal dose damage simulations to adjust the real-time thermal monitoring system. In some implementations, a reconstructed real-time thermal map may provide feedback to a computational simulation to correct for the model parameters.

FIG. 9B is a diagram depicting how intra-operative data can be regarded as an input of a thermal computational simulation to allow model parameter estimation and provide improved real-time thermal monitoring.

FIG. 9C is a diagram of an example model to obtain improved temperature monitoring during thermal ablation. In some implementations, the model may include a thermal propagation model that is associated with an ultrasound simulation tool to simulate intra-operative quantities (e.g., time of flight, brightness (B-mode) images, attenuation, speed of sound, and/or the like). Simulated quantities may be compared with actual measurements that are obtained intra-operatively. In some implementations, an optimization model may be utilized to estimate parameters of the thermal propagation model to provide an updated real-time thermal monitoring.

FIG. 9D is a diagram depicting how a thermal propagation simulation can be used to improve thermal map reconstruction from intra-operative ultrasound measurements as pattern injection or ultrasound time of flight acquisition. In some implementations, one or more tools that enable real-time assessment of thermal dose distribution, via an integrative approach based on both intra-operative ToF measurements and patient-specific simulation methods, may be utilized.

FIG. 9E is a diagram of an example patient-specific simulation method. In some implementations, a physics-based model may simulate thermal maps (e.g., as shown in part (c) of FIG. 9E). The thermal maps may be utilized to solve an ill-posed limited-angle tomography problem (e.g., as shown in part (b) of FIG. 9E) by providing a priori knowledge and reducing a number of unknowns to solve X (e.g., slowness). In some implementations, a least squares model, a Poisson model, and/or the like may be employed. The reconstructed thermal images, as well as intraoperative ToF measurements (e.g., as shown in part (a) of FIG. 9E), may be used to personalize a patient-specific model. At a next time step, a new FUS simulation may be executed with updated patient-specific values of biophysical parameters, providing more accurate initializations to solve the limited-angle tomographic problem.

The relationship between temperature and speed-of-sound (SoS) enables ultrasound thermometry through tomographic reconstruction of SoS maps from direct ToF measures. However, a tomographic problem is rank deficient, which may lead to multiple solutions. More specifically, recorded ToF data may be sparse, as a maximum number of equations may equal a number of FUS elements multiplied by a number of receivers employed. In some implementations, the SoS may be reconstructed at a voxel level in order to address the tomographic problem. Furthermore, a relationship between a change in temperature and SoS may be linear until a certain point, and this relationship may be tissue-specific. In some implementations, combining advanced quantitative tomographic imaging, and patient-specific simulation incorporating prior knowledge of biological and physical phenomena in thermal ablation, may address the relationship problem.

For example, in some implementations, limited angle tomography may be utilized. Often used in x-ray mammography, and referred to as tomosynthesis, limited angle tomography has a highly transformed routine screening, enabling quantitative 3D imaging with significantly enhanced value. In transmission ultrasound imaging (e.g., in contrast to reflection imaging in B-mode), the transmitter and receiver transducers may be located at different known positions with respect to a volume of interest. This ToF approach is affected less by directivity of a receiver element than in a pulse-echo scheme. A received signal can be used to reconstruct acoustic properties of a volume, such as SoS, attenuation, and/or the like.

In some implementations, optimal probe placement and image reconstruction are two important considerations to achieve effective in vivo ultrasound tomosynthesis. In such implementations, extensive phantom studies involving anthropomorphic digital phantoms and experimental phantoms may be performed. Phantoms studies may narrow down data acquisition parameters and improve reconstruction algorithms, which are then followed up by real subject studies.

In some implementations, to address the SoS reconstruction problem, a time-of-flight technique (e.g., a ray-based technique) may be utilized. For example, given a grid of pixels, a system matrix S may be used to describe how much a given ray travels through each pixel of the grid. Each row of the system matrix may correspond to one ray and may contain path lengths corresponding to each grid cell. The system matrix S may be constructed based on a Siddon method, and may be further refined using more advanced interpolations (e.g., splines). The SoS can be reconstructed by ToF measurements for all rays. The ToF measurements may be represented by a vector of length N_t (e.g., a number of possible rays to the receivers), and an image, X, may be represented by a vector of length N_g (e.g., a number of voxels). Instead of directly calculating the speed, an inverse of the speed (e.g., referred to as slowness) may be calculated. A time required for an ultrasound signal to travel along a ray may be equal to a sum of a time to pass through all cells, and a time t required to travel through one cell may be t=s*x, where s is the path length along the cell, and x is the cell's slowness. This leads to solving the following equation for X:SX=ToF, where S is the system matrix and X is an image vector. In some implementations, analytical methods (e.g., pseudo-inverse), iterative methods (e.g., conjugate gradient), statistical methods (e.g., expectation maximization), and/or the like may be utilized. Iterative and statistical image generation may be considerably more quantitative, but more time consuming.

In some implementations, to address the SoS reconstruction problem, a wavefront tomography method may be utilized. The waveform tomography method can improve results (e.g., as used in breast imaging and past efforts of geophysics researchers on seismic waveform inversion). For the methods to address the SoS reconstruction problem, Bayesian maximum a posteriori (MAP) frameworks can also be appended, including sparse tomographic reconstruction, utilizing compressed sensing involving L1-norm minimization.

In some implementations, an attenuation coefficient, which may also convey information about temperature (e.g., especially when linked with SoS) may be reconstructed. The above reconstruction methods may be used to reconstruct the attenuation coefficient. In such implementations, an original transmitter signal may be known, and a detected signal intensity may define a measurement, which may be tomographically reconstructed to generate attenuation maps.

In some implementations, prior knowledge of biological and physical phenomena involved in thermal ablation may be utilized. Via the usage of computational models, heat diffusion and cellular necrosis may be simulated. The prior knowledge may include developing computational models of radiofrequency ablation (RFA), which may be evaluated against pre-clinical and clinical data of subjects with tumors.

FIG. 9F is a diagram depicting a predictive power of a radiofrequency ablation model. In some implementations, the model may simulate a lesion created by RFA (the portion labeled “simulated lesion”), which may be similar to a ground truth lesion (the portion labeled “post-op lesion”) segmented on a postoperative MR image and registered to a pre-operative image. As shown in FIG. 9F, the simulation may predict that the lesion does not entirely cover a tumor.

In some implementations, FIG. 9F depicts results of computational modeling of RFA. The simulated necrotic lesions may compare qualitatively and quantitatively well with the lesions observed on the patient images acquired after ablation. Taking into account blood flow and estimating parameters is also highlighted. From a pre-operative 3D anatomical image and given some biophysical parameters, such as an applied input temperature, an expected temperature evolution during the ablation can be simulated with a temporal resolution, a spatial resolution, and/or the like, and the expected temperature evolution may also be simulated based on varying properties from one tissue part to another (e.g. blood vessels versus parenchyma versus tumor tissue in the liver).

In some implementations, these patient-specific simulations may be coupled with tomographic image reconstruction. Expected thermal maps can be used to reduce a number of SoS unknowns in a region of interest (ROI) around an ablation focal point by grouping together, in a same layer, voxels which are expected to have a same temperature according to the patient-specific simulation.

In some implementations, generating thermal maps from the SoS maps, the attenuation maps, and/or the like could lead to a range of solutions. However, using a patient-specific simulation, solutions may be distinguished because a temporal and spatial temperature evolution should be smooth (e.g., there should not be a significant jump in temperature from one time point to a next time point, from a particular spatial point to an adjacent spatial point, and/or the like). Furthermore, the patient-specific simulation may be used to distinguish solutions because temperature should decrease farther away from the ablation focal point. In some implementations, the SoS reconstruction problem may be addressed by initialization based on patient-specific thermal maps.

In some implementations, limited angle reconstruction of speed-of-sound may be developed based on direct ToF measurements. These measurements may be validated within ±3° C. with MRI thermometry up to 55° C.

FIG. 9G is a diagram depicting how a framework can be implemented so that a deep learning engine may enable HIFU guidance. In some implementations, FIG. 9G depicts performance of MR-guided HIFU at a same time as ultrasound thermal monitoring. Data may be collected, including time-of-flight and attenuation measurements, tracking of a PZT element, and/or the like in order to determine a relative position of the PZT element relative to HIFU elements. Simulation may be utilized to generate synthetic images by adding extra simulated features as input of the deep learning engine. Furthermore, the deep learning engine may generate thermal images, which may enable HIFU guidance at a lower cost and at a larger number of treatment settings because MRI equipment is not required.

In some implementations, machine learning may be utilized to generate synthetic images from ultrasound data. A machine learning model may be utilized to synthetize thermal images using ultrasound acquisition. This may provide a simple and low-cost system with thermal images at a high frame rate and without the requirement of an MRI scanner.

In some implementations, thermal images may be reconstructed based on a machine learning model. Due to ultrasound physics, ultrasound signals may change as a target is heated (e.g., during a HIFU ablation treatment), cooled (e.g., during a cryoablation treatment), and/or the like. With MR thermal images and corresponding ultrasound signals, a machine learning model can be trained. The training information may include ultrasound channel data, B-mode images, time of flight data, ultrasound elements locations, and/or other ultrasound information. By detecting a change of these ultrasound signal properties during ablation, a thermal map can be recovered through the machine learning model.

FIG. 9H is a diagram of an example framework for simulation-driven monitoring of HIFU ablation. In some implementations, a HIFU simulation may be performed based on nonlinear ultrasound propagation using a k-space model coupled with thermal propagation in biological tissue using a reaction-diffusion equation. Using the speed of sound (SoS) dependency on temperature, ultrasound pressure waves traveling through the ablation zone and carrying time-of-flight (ToF) information may be modeled. An active ultrasound element may be fabricated to receive the intraoperative information. For monitoring, actual ToF measurements (intra-operative data) may be compared to expected ToF measurements from a HIFU simulation (simulated intra-operative data). If similar, the ablation is going as expected and simulated thermal maps can be used. Otherwise, the ablation should stop. In this way, insufficient ablation in the target region and unexpected off-target ablation are both avoided.

In some implementations, intra-operative ultrasound measurements may be utilized to personalize biophysical parameters involved in the simulation. A patient-specific model may enable simulating a delay that is expected to affect different ToF data due to the ablation evolution. Therefore, biophysical model parameters can be optimized by minimizing an error between the measured ToF and the simulated ToF, and between the reconstructed thermal maps and the simulated thermal maps. Modeling can be performed in real-time or faster than real-time with a Lattice Boltzmann method. In some implementations, an iterative loop between SoS image reconstruction and patient-specific simulation may be utilized in order to generate more accurate 3D thermal images. Limited SoS maps at the regional level from direct ToF measurements may be utilized.

FIG. 9I is a diagram of an example experimental results from implementing and testing the second example method 900 on phantom data. Portion (A) of FIG. 9I depicts measured (top) and simulated (bottom) ToF data from one HIFU element to an active US element. In some implementations, delays of 0.1, 0.2, 0.3 microsecond(s) may occur after the first, second, and third heating interval(s). Portion (B) of FIG. 9I depicts an MR thermal image (top) and a simulated thermal map (bottom) that compare well centered around a targeted region of interest.

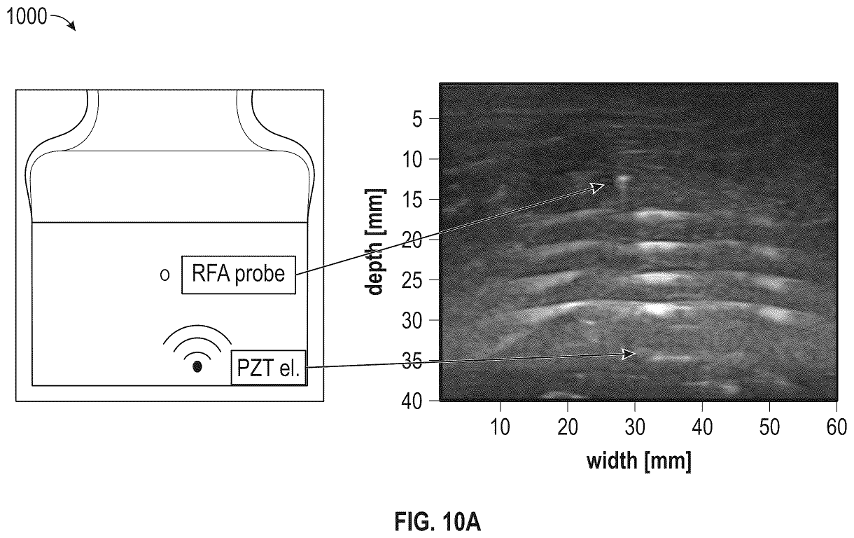

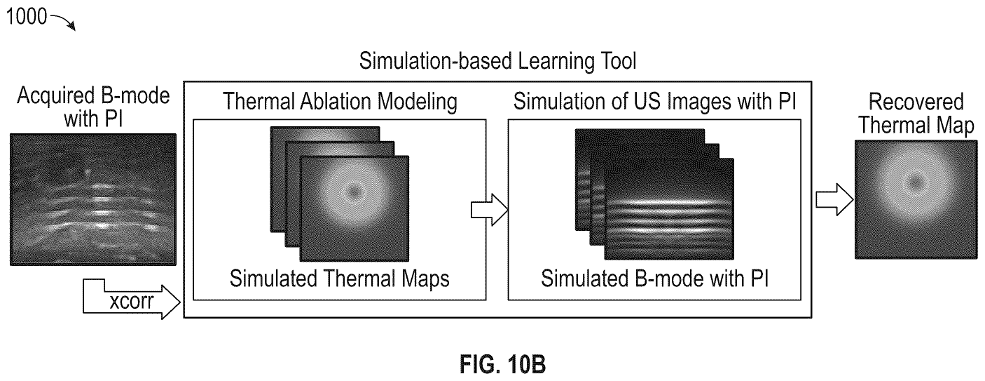

In some implementations, FIGS. 10A and 10B depict a third example method 1000 that may be implemented in one or more systems and/or devices described elsewhere herein (e.g., systems and/or devices described above in connection with FIGS. 1-7). The third example method 1000 may include reconstruction of temperature change by analyzing injected pattern deformation.

In the third example method 1000, a virtual pattern may be injected in an ultrasound B-mode image. In some implementations, the pattern may be generated using an ultrasound machine or a HIFU system.

In some implementations, a certain ultrasound pattern can be injected in the ultrasound B-mode image before ablation. Due to an increase in temperature, acoustic properties such as the speed of sound, attenuation, and/or the like may vary, whereby the injected pattern may deform from an initial state. Thermal maps and thus thermal dosage can be reconstructed by tracking these changes in the injected patterns and thus tracking the acoustic property changes. Learning tools based on deep learning, machine learning, simulation of synthetic images, and/or the like may be utilized to determine an effect of temperature changes on the virtual injection pattern, and thus enable recovery of thermal maps and a delivered thermal dose. This learning process can be achieved with simulation and/or actual data acquisition.

FIG. 10A is a diagram of an example experiment that has been performed to illustrate the concept described above. The left portion of FIG. 10A depicts an experimental setup where an ultrasound imaging probe is placed on top of tissue, and the right portion of FIG. 10A depicts a B-mode image acquired during the ablation (e.g., an RFA probe, an active PZT element, and a virtual pattern injection (PI) may be visible in the B-mode image).

In some implementations, the imaging method for temperature monitoring may be based on the injection of the virtual ultrasound pattern in the ultrasound brightness mode (B-mode) image coupled with biophysical simulation of heat propagation. This imaging method does not require any hardware extensions to an ultrasound B-mode system. The imaging method may establish a bi-directional ultrasound communication between an ultrasound imaging machine and an active element inserted within the tissue. A virtual pattern can then directly be created in the ultrasound B-mode display during the ablation by controlling a timing and an amplitude of the ultrasound field generated by the active element. Changes of the injected pattern are related to the change of the ablated tissue temperature through the additional knowledge of a biophysical model of heat propagation in the tissue. Such changes may be monitored during ablation and used to generate spatially and temporally accurate thermal maps.

FIG. 10B is a diagram depicting method feasibility and applicability on a clinical ultrasound scanner using ex vivo data. In some implementations, FIG. 10B depicts ultrasound B-mode images with a PI that are simulated with corresponding thermal maps using a biophysical thermal ablation model to constitute a simulation-based learning tool. As a new B-mode with PI is acquired during ablation, the new B-mode is evaluated with a learning tool to recover a corresponding thermal map. The learning tool may include a machine learning model, a deep learning model, and/or the like that is trained with training data. The training data can be created by physics-based and ultrasound simulation, collected with biological tissues, and/or the like.

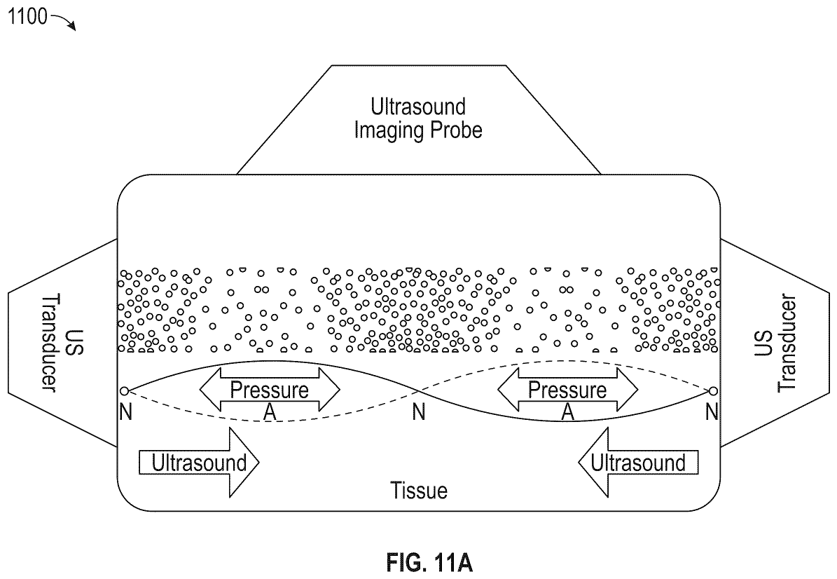

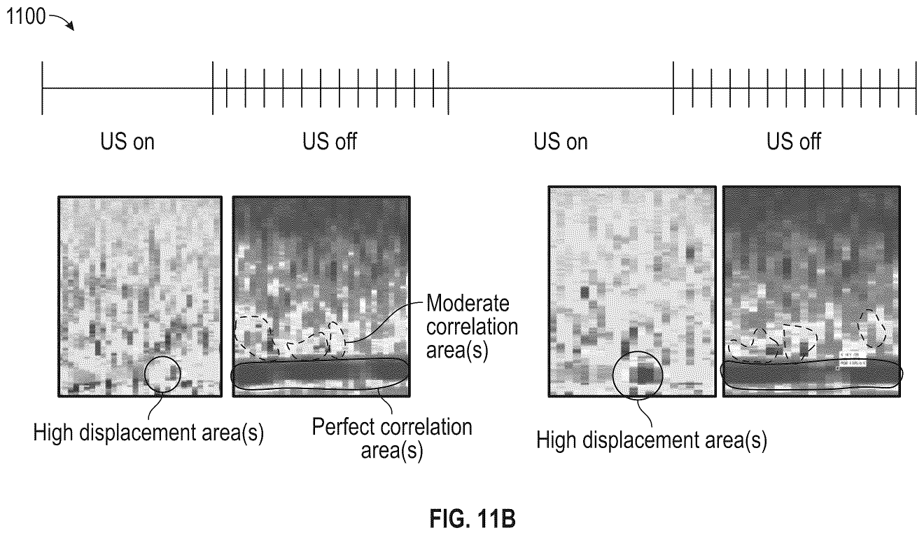

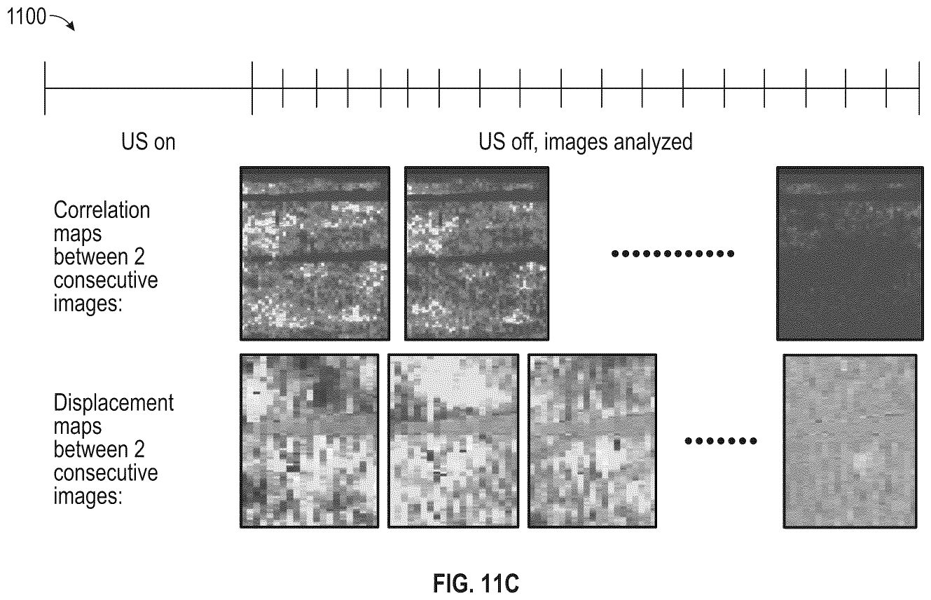

In some implementations, FIGS. 11A-11C depict a fourth example method 1100 that may be implemented in one or more systems and/or devices described elsewhere herein (e.g., systems and/or devices described above in connection with FIGS. 1-7). The fourth example method 1100 may include ultrasound standing wave elastography.

In the fourth example method 1100, a thermal dose may be measured with ultrasound standing wave elastography. An ultrasound elastography image may be acquired by applying stress to the tissue and measuring a local displacement and deformation, from which a tissue stiffness map can be derived. There are various methods to apply stress to tissue, such as free hand palpation, vibration, acoustic radiation force (ARF), shear wave (SW), and/or the like. However, these methods typically cannot generate a fully controllable, programmable, and stable stress in the tissue. Standing wave, also known as a stationary wave, is a wave that stays in a constant position. A standing wave can be generated when two waves traveling in opposite directions have identical frequencies, amplitudes, and beam paths. As a result, a node and antinode of a combined wave may remain at a same position. An ultrasound wave is a longitudinal wave, which means that antinodes have higher pressure than the nodes, and the pressure is along the wave traveling direction. If an ultrasound standing wave is formed in the tissue, because the antinodes have higher pressure than the nodes, tissue will be pushed from the higher-pressure region to the lower-pressure region, and physical displacements and deformations may be formed, which can be used for the elastography imaging.

FIG. 11A is a diagram of an example configuration of standing wave elastography. As shown in the FIG. 11A, using two ultrasound transducers, a periodic stress pattern can be generated in the tissue and monitored by an ultrasound imaging probe. A more complex pattern can be generated using multiple transducers. Because of the nature of the standing wave, the generated pattern may be stationary. The pattern may be determined by a transducer frequency phase and tissue sound speed. By tuning the phase, frequency, and amplitude, a stress pattern can be fully programmable and dynamic.

Aside from the elastography, the fourth example method 1100 may be utilized for speed of sound map measurement. Given a wave frequency, a wave length is determined by the speed of sound. A standing wave pattern may freeze a wave and make a direct measurement of wavelength possible.

FIG. 11B is a diagram of an example experimental result of a plastisol phantom. As shown, displacement maps (left of each pair) and correlation maps (right of each pair) between the first and last images may be analyzed. Relatively high displacement areas (e.g., encircled areas in the displacement maps) are repeated in subsequent cycles, in areas of high correlation (e.g., areas encircled in solid lines indicate areas of perfect correlation, and areas encircled in dotted lines indicate areas of moderate correlation, such as a correlation of 0.5).

FIG. 11C is a diagram of an example relaxation process of a gelatin phantom in an ultrasound standing wave off period. As shown, the displacement and correlation maps are generated from consecutive images during the ultrasound standing wave off period. The result may indicate that by an end of an ultrasound standing wave on period, a phantom local displacement may reach a maximum. Once the standing wave is turned off, a quasi-static acoustic pressure disappears, so the phantom material may relax to an original state.

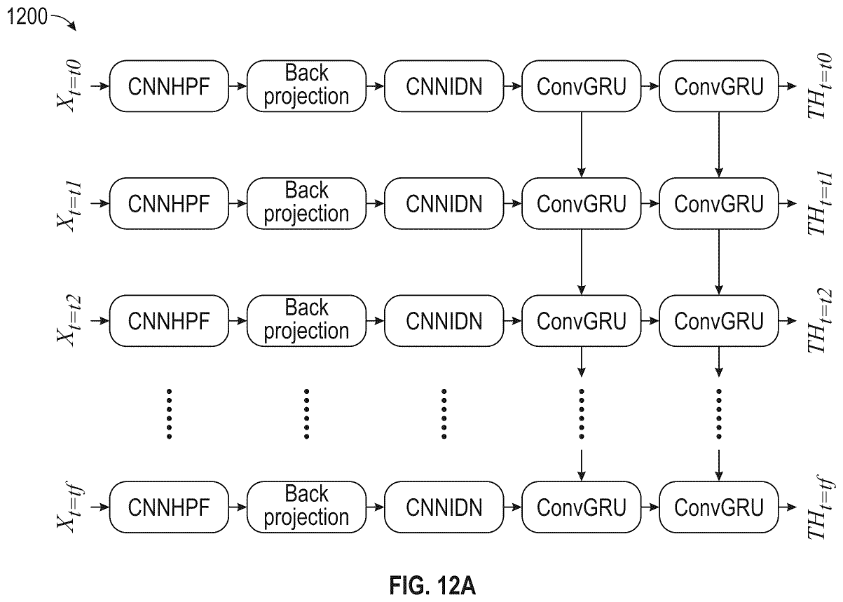

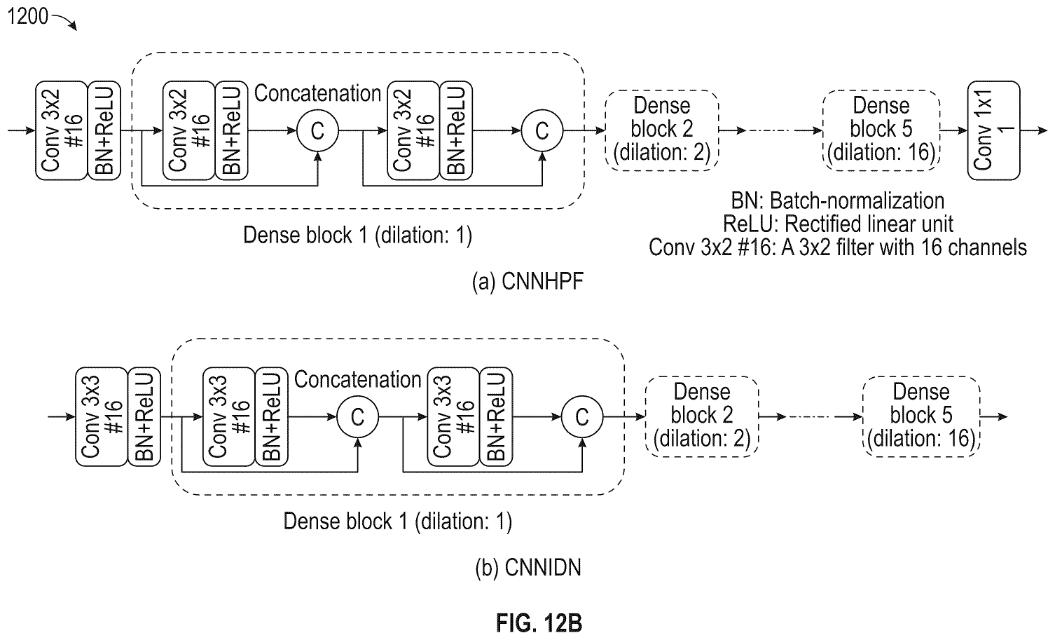

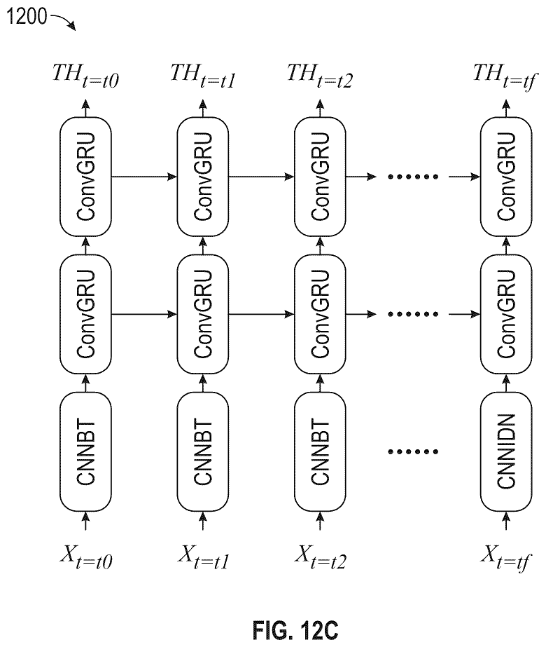

FIGS. 12A-12C depict a fifth example method 1200 that may be implemented in one or more systems and/or devices described elsewhere herein (e.g., systems and/or devices described above in connection with FIGS. 1-7). The fifth example method 1200 may include application of deep learning in estimation of thermal images from a set of ultrasound signals.

Deep learning based inverse problems (or image reconstruction) can be broadly categorized into two groups. A first group uses an end-to-end deep neural network to solve the problem. Instead of completely relying on neural networks, a second group uses a standard image reconstruction approach (e.g., filtered back projection) for an initial reconstruction followed by exploiting a deep neural network to correct the reconstruction artifacts.

In some implementations, a deep neural network-based approach may be utilized to estimate spatial and temporal temperature distribution from a set of ultrasound signals. Such implementations may integrate a standard physics-based method, convolutional neural networks, and recurrent neural networks.

There may be two possible scenarios in a thermal monitoring setup. The first scenario includes acquiring RF signals using a piezoelectric element or an ultrasound transducer. In the second scenario, a B-mode image may be obtained based on pattern injection (e.g., as shown in FIG. 10A) or B-mode standing wave elastography (e.g., as shown in FIG. 11A). For the first scenario, time-of-flight (ToF), attenuation, and/or the like may be computed from the acquired RF signals that are provided as inputs to the deep neural network. In contrast, for the second scenario, the network may take the B-mode image as input.

For the first scenario, a filtered back-projection model may be utilized, where a standard high-pass filtering may be replaced before the back-projection by a deep convolutional neural network (CNN). FIG. 12A depicts a deep neural network that may be used to estimate the thermal map from a set of RF signals. The network takes the estimated ToF, attenuation, and/or other photoacoustic data as input and produces a two-dimensional thermal map as output. A set of convolutional layers followed by non-linear activations may be utilized for an effective filtering before the back-projection. This is referred to as CNNHPF, where HPF stands for high-pass filtering. Portion (a) of FIG. 12B depicts a detailed architecture of CNNHPF. CNNHPF may include five dense blocks, where each dense block includes two densely connected convolutional layers. In principle, a dense convolutional layer uses all of the previous features as input, and therefore allows an effective feature propagation while eliminating a vanishing gradient problem. A difference among five dense blocks lies in an amount of dilation used in the convolutional layer. A convolution with a dilation enables a convolutional filtering with higher receptive field size without decreasing the resolution subsequently using the same number of parameters.

After filtering by CNNHPF, a physics-based back-projection technique may be utilized for reconstruction. The back-projection may be based on a bent-ray model that provides a good approximation of ultrasound wave propagation in a heterogenous medium. After the back-projection, a post CNN may be utilized to eliminate the artifacts due to partial reconstruction from a limited angle tomography. This CNN is indicated as CNNIDN in part (b) of FIG. 12B, where IDN stands for image de-noising. The architecture of CNNIDN has a similar structure of CNNHPF. However, a convolutional kernel in CNNHPF may include a size of 3×2, where the convolutional kernel in CNNIDN may include a size of 3×3. A dilation is applied along the first dimension in CNNHPF and dilations are applied along both dimensions in CNNIDN, and there is no 1×1 convolution performed at the end in CNNIDN.

A recurrent neural network (RNN) may be utilized at an end, as shown in FIG. 12A, to exploit temporal information across a set of temporally acquired ultrasound signals to predict a thermal map. Several improvements of RNN, e.g. long-short-term-memory (LSTM) or gated recurrent unit (GRU), may be utilized. GRU requires low memory and has shown similar performance compared to LSTM.

ConvGRU is an extension of GRU to extract spatial temporal features among a series of 2D feature maps. ConvGRU may take a current input Xₜ and previous hidden state Hₜ₋₁, and may generate a current hidden state Hₜ. A relation of Hₜ with Xₜ and Hₜ₋₁ is given below:

$${Z}_{t} = σ ( {W}_{xz} * {X}_{t} + {W}_{hz} * {H}_{t - 1} )$$

$${R}_{t} = σ ( {W}_{xr} * {X}_{t} + {W}_{hr} * {H}_{t - 1} )$$

$${\tilde{H}}_{t} = tanh ( {W}_{xh} * {X}_{t} + {W}_{hh} * ( {R}_{t} ∘ {H}_{t - 1} ) )$$

$${H}_{t} = ( 1 - {Z}_{t} ) ∘ {H}_{t - 1} + {Z}_{t} ∘ {\tilde{H}}_{t}$$

where * and ° represent convolution and element-wise matrix multiplication, respectively, W_{xz}, W_{hz}, Wₓᵣ, Wₕᵣ, Wₓₕ, Wₕₕ are the parameters to be learned during training, σ represents the sigmoid operation, and tanh is the activation function.

FIG. 12C is a diagram of an example architecture of a deep neural network for predicting thermal maps from a set of given B-mode images. In some implementations, CNN (CNNBT, BT for B-mode to temperature) may be utilized at the beginning to convert the B-mode images to a thermal map, and a ConvGRU may be utilized at the end to estimate the thermal map.

As indicated above, FIGS. 8A-12C are provided merely as one or more examples. Other examples may differ from what is described with regard to FIGS. 8A-12C.

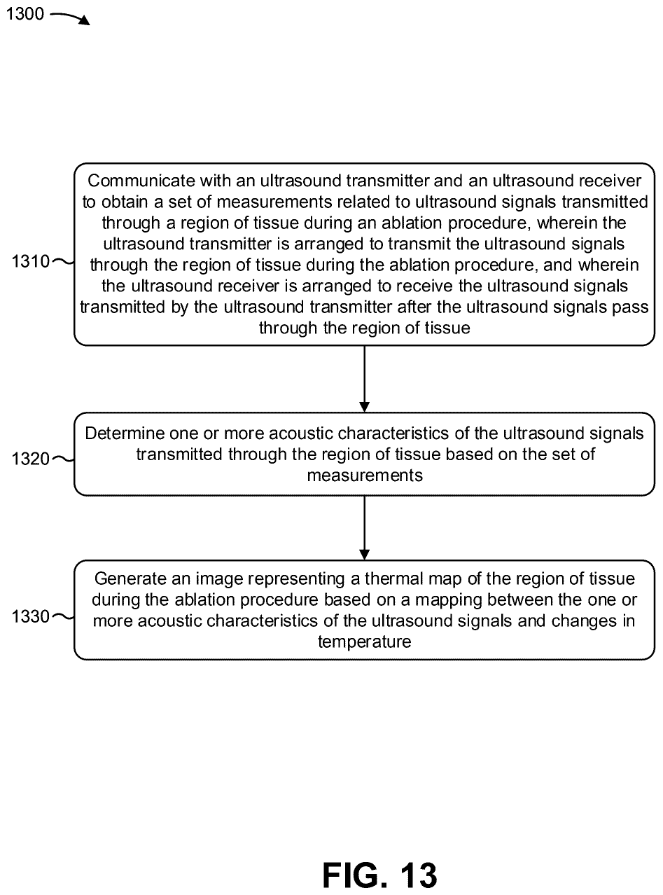

FIG. 13 is a flow chart of an example process 1300 for real-time ultrasound monitoring for ablation therapy. In some implementations, one or more process blocks of FIG. 13 may be performed by a signal processor (e.g., signal processor 102). In some implementations, one or more process blocks of FIG. 13 may be performed by another device or a group of devices separate from or including the signal processor, such as an ultrasound transceiver (e.g., ultrasound transceiver 101), a high-intensity focused ultrasound (HIFU) element (e.g., HIFU element 103), a function generator and/or amplifier (e.g., function generator and/or amplifier 104), a HIFU system (e.g., HIFU system 201, 403), an ultrasound receiver (e.g., ultrasound receiver 202), a control system (e.g., control system 205, 305), an ablation device (e.g., ablation device 301), an ultrasound transducer (e.g., ultrasound transducer 302), an ablation control system (e.g., ablation control system 303), an active PZT control system (e.g., active PZT control system 304), an ultrasound probe (e.g., ultrasound probe 306, 401), an ultrasound element control system (e.g., ultrasound element control system 600), a pulser block (e.g., pulser block 610), a data collection block (e.g., data collection block 620), a signal processing unit (e.g., signal processing unit 630), and/or the like.

As shown in FIG. 13, process 1300 may include communicating with an ultrasound transmitter and an ultrasound receiver to obtain a set of measurements related to ultrasound signals transmitted through a region of tissue during an ablation procedure, wherein the ultrasound transmitter is arranged to transmit the ultrasound signals through the region of tissue during the ablation procedure, and wherein the ultrasound receiver is arranged to receive the ultrasound signals transmitted by the ultrasound transmitter after the ultrasound signals pass through the region of tissue (block 1310). For example, the signal processor (e.g., using processor 720, memory 730, storage component 740, input component 750, output component 760, communication interface 770, and/or the like) may communicate with an ultrasound transmitter and an ultrasound receiver to obtain a set of measurements related to ultrasound signals transmitted through a region of tissue during an ablation procedure, as described above. In some implementations, the ultrasound transmitter may be arranged to transmit the ultrasound signals through the region of tissue during the ablation procedure, and the ultrasound receiver may be arranged to receive the ultrasound signals transmitted by the ultrasound transmitter after the ultrasound signals pass through the region of tissue.

As further shown in FIG. 13, process 1300 may include determining one or more acoustic characteristics of the ultrasound signals transmitted through the region of tissue based on the set of measurements (block 1320). For example, the signal processor (e.g., using processor 720, memory 730, storage component 740, input component 750, output component 760, communication interface 770, and/or the like) may determine one or more acoustic characteristics of the ultrasound signals transmitted through the region of tissue based on the set of measurements, as described above.

As further shown in FIG. 13, process 1300 may include generating an image representing a thermal map of the region of tissue during the ablation procedure based on a mapping between the one or more acoustic characteristics of the ultrasound signals and changes in temperature (block 1330). For example, the signal processor (e.g., using processor 720, memory 730, storage component 740, input component 750, output component 760, communication interface 770, and/or the like) may generate an image representing a thermal map of the region of tissue during the ablation procedure based on a mapping between the one or more acoustic characteristics of the ultrasound signals and changes in temperature, as described above.

Process 1300 may include additional implementations, such as any single implementation or any combination of implementations described below and/or in connection with one or more other processes described elsewhere herein.

In a first implementation, the one or more acoustic characteristics may include changes in one or more of speeds or intensities at which the ultrasound signals travel through the region of tissue. In some implementations, the mapping used to generate the image representing the thermal map of the region of tissue may be based on a relationship between changes in temperature and the changes in the speeds or intensities at which the ultrasound signals travel through the region of tissue.

In a second implementation, alone or in combination with the first implementation, the mapping used to generate the image representing the thermal map of the region of tissue may be further based on temperature-dependent variations in a time of flight, an attenuation, a phase, and/or a nonlinearity for at least one of the ultrasound signals transmitted through the region of tissue.

In a third implementation, alone or in combination with one or more of the first and second implementations, the ultrasound receiver may include a transducer array having one or more transducer elements with known locations, and the signal processor may determine a relative geometry between the ultrasound transmitter and the ultrasound receiver based on the known locations of the one or more transducer elements and time of flight data associated with ultrasound signals transmitted from the ultrasound transmitter to the ultrasound receiver before the ablation procedure.

In a fourth implementation, alone or in combination with one or more of the first through third implementations, the signal processor may generate the image representing the thermal map of the region of tissue using one or more tomographic techniques based on the relative geometry between the ultrasound transmitter and the ultrasound receiver.

In a fifth implementation, alone or in combination with one or more of the first through fourth implementations, the signal processor, when generating the thermal map of the region of tissue, may use a thermal propagation model to segment the region of tissue into groups of voxels that have similar temperatures and reduce a region of interest to be represented by the thermal map to an area where the ultrasound signals are causing a change in temperature during the ablation procedure based on the groups of voxels that have the similar temperatures.

In a sixth implementation, alone or in combination with one or more of the first through fifth implementations, the signal processor may obtain patient-specific simulation data including an expected temperature evolution for the region of tissue during the ablation procedure based on a three-dimensional anatomical image of the region of tissue and one or more biophysical parameters and use the patient-specific simulation data including the expected temperature evolution for the region of tissue in combination with one or more tomographic image reconstruction techniques to generate the image representing the thermal map of the region of tissue.

In a seventh implementation, alone or in combination with one or more of the first through sixth implementations, the signal processor may obtain simulation data including a simulated thermal map based on expected time of flight measurements for the ultrasound signals to be transmitted through the region of tissue during the ablation procedure and perform an action based on a comparison of actual time of flight measurements for the ultrasound signals transmitted through the region of tissue during the ablation procedure and the expected time of flight measurements for the ultrasound signals.

Although FIG. 13 shows example blocks of process 1300, in some implementations, process 1300 may include additional blocks, fewer blocks, different blocks, or differently arranged blocks than those depicted in FIG. 13. Additionally, or alternatively, two or more of the blocks of process 1300 may be performed in parallel.

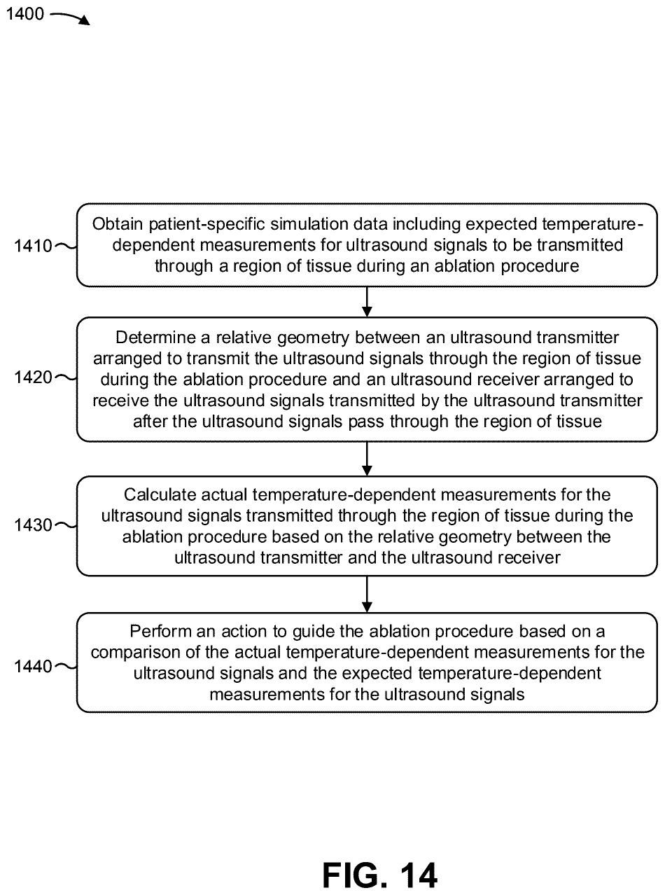

FIG. 14 is a flow chart of an example process 1400 for real-time ultrasound monitoring for ablation therapy. In some implementations, one or more process blocks of FIG. 14 may be performed by a signal processor (e.g., signal processor 102). In some implementations, one or more process blocks of FIG. 14 may be performed by another device or a group of devices separate from or including the signal processor, such as an ultrasound transceiver (e.g., ultrasound transceiver 101), a high-intensity focused ultrasound (HIFU) element (e.g., HIFU element 103), a function generator and/or amplifier (e.g., function generator and/or amplifier 104), a HIFU system (e.g., HIFU system 201, 403), an ultrasound receiver (e.g., ultrasound receiver 202), a control system (e.g., control system 205, 305), an ablation device (e.g., ablation device 301), an ultrasound transducer (e.g., ultrasound transducer 302), an ablation control system (e.g., ablation control system 303), an active PZT control system (e.g., active PZT control system 304), an ultrasound probe (e.g., ultrasound probe 306, 401), an ultrasound element control system (e.g., ultrasound element control system 600), a pulser block (e.g., pulser block 610), a data collection block (e.g., data collection block 620), a signal processing unit (e.g., signal processing unit 630), and/or the like.

As shown in FIG. 14, process 1400 may include obtaining patient-specific simulation data including expected temperature-dependent measurements for ultrasound signals to be transmitted through a region of tissue during an ablation procedure (block 1410). For example, the signal processor (e.g., using processor 720, memory 730, storage component 740, input component 750, output component 760, communication interface 770, and/or the like) may obtain patient-specific simulation data including expected temperature-dependent measurements for ultrasound signals to be transmitted through a region of tissue during an ablation procedure, as described above.

As further shown in FIG. 14, process 1400 may include determining a relative geometry between an ultrasound transmitter arranged to transmit the ultrasound signals through the region of tissue during the ablation procedure and an ultrasound receiver arranged to receive the ultrasound signals transmitted by the ultrasound transmitter after the ultrasound signals pass through the region of tissue (block 1420). For example, the signal processor (e.g., using processor 720, memory 730, storage component 740, input component 750, output component 760, communication interface 770, and/or the like) may determine a relative geometry between an ultrasound transmitter arranged to transmit the ultrasound signals through the region of tissue during the ablation procedure and an ultrasound receiver arranged to receive the ultrasound signals transmitted by the ultrasound transmitter after the ultrasound signals pass through the region of tissue, as described above.

As further shown in FIG. 14, process 1400 may include calculating actual temperature-dependent measurements for the ultrasound signals transmitted through the region of tissue during the ablation procedure based on the relative geometry between the ultrasound transmitter and the ultrasound receiver (block 1430). For example, the signal processor (e.g., using processor 720, memory 730, storage component 740, input component 750, output component 760, communication interface 770, and/or the like) may calculate actual temperature-dependent measurements for the ultrasound signals transmitted through the region of tissue during the ablation procedure based on the relative geometry between the ultrasound transmitter and the ultrasound receiver, as described above.

As further shown in FIG. 14, process 1400 may include performing an action to guide the ablation procedure based on a comparison of the actual temperature-dependent measurements for the ultrasound signals and the expected temperature-dependent measurements for the ultrasound signals (block 1440). For example, the signal processor (e.g., using processor 720, memory 730, storage component 740, input component 750, output component 760, communication interface 770, and/or the like) may perform an action to guide the ablation procedure based on a comparison of the actual temperature-dependent measurements for the ultrasound signals and the expected temperature-dependent measurements for the ultrasound signals, as described above.

Process 1400 may include additional implementations, such as any single implementation or any combination of implementations described below and/or in connection with one or more other processes described elsewhere herein.

In a first implementation, the patient-specific simulation data may further include a simulated thermal map based on the expected temperature-dependent measurements for the ultrasound signals, and the action may include displaying the simulated thermal map to guide the ablation procedure based on the comparison indicating a threshold similarity between the actual temperature-dependent measurements and the expected temperature-dependent measurements for the ultrasound signals.

In a second implementation, alone or in combination with the first implementation, the action may include causing the ablation procedure to stop based on the comparison indicating one or more of insufficient ablation in a targeted area of the region of tissue or off-target ablation in the region of tissue.

In a third implementation, alone or in combination with one or more of the first and second implementations, the patient-specific simulation data may further include an expected temperature evolution for the region of tissue during the ablation procedure based on a three-dimensional anatomical image of the region of tissue and one or more biophysical parameters, and the action may include using the expected temperature evolution for the region of tissue in combination with one or more tomographic image reconstruction techniques to generate an image representing a thermal map of the region of tissue during the ablation procedure.

In a fourth implementation, alone or in combination with one or more of the first through third implementations, the expected temperature evolution for the region of tissue may be represented according to one or more of a temporal resolution or a spatial resolution.

In a fifth implementation, alone or in combination with one or more of the first through fourth implementations, the image may be a synthesized thermal image generated using one or more of a deep learning technique or a machine learning technique.

In a sixth implementation, alone or in combination with one or more of the first through fifth implementations, the image may be an ultrasound elastography image based on pressure changes along the ultrasound signals transmitted through the region of tissue.

Although FIG. 14 shows example blocks of process 1400, in some implementations, process 1400 may include additional blocks, fewer blocks, different blocks, or differently arranged blocks than those depicted in FIG. 14. Additionally, or alternatively, two or more of the blocks of process 1400 may be performed in parallel.

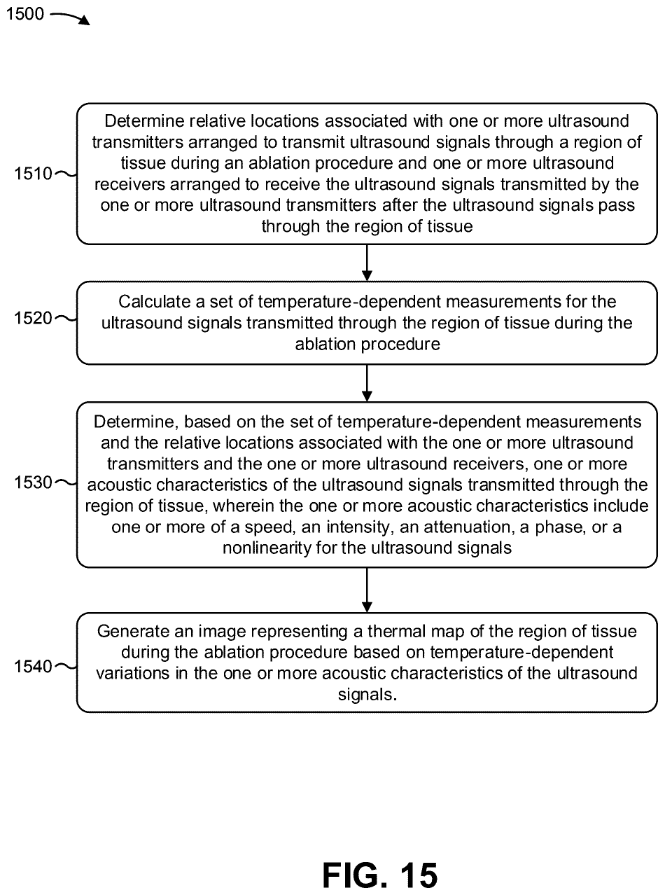

FIG. 15 is a flow chart of an example process 1500 for real-time ultrasound monitoring for ablation therapy. In some implementations, one or more process blocks of FIG. 15 may be performed by a signal processor (e.g., signal processor 102). In some implementations, one or more process blocks of FIG. 15 may be performed by another device or a group of devices separate from or including the signal processor, such as an ultrasound transceiver (e.g., ultrasound transceiver 101), a high-intensity focused ultrasound (HIFU) element (e.g., HIFU element 103), a function generator and/or amplifier (e.g., function generator and/or amplifier 104), a HIFU system (e.g., HIFU system 201, 403), an ultrasound receiver (e.g., ultrasound receiver 202), a control system (e.g., control system 205, 305), an ablation device (e.g., ablation device 301), an ultrasound transducer (e.g., ultrasound transducer 302), an ablation control system (e.g., ablation control system 303), an active PZT control system (e.g., active PZT control system 304), an ultrasound probe (e.g., ultrasound probe 306, 401), an ultrasound element control system (e.g., ultrasound element control system 600), a pulser block (e.g., pulser block 610), a data collection block (e.g., data collection block 620), a signal processing unit (e.g., signal processing unit 630), and/or the like.

As shown in FIG. 15, process 1500 may include determining relative locations associated with one or more ultrasound transmitters arranged to transmit ultrasound signals through a region of tissue during an ablation procedure and one or more ultrasound receivers arranged to receive the ultrasound signals transmitted by the one or more ultrasound transmitters after the ultrasound signals pass through the region of tissue (block 1510). For example, the signal processor (e.g., using processor 720, memory 730, storage component 740, input component 750, output component 760, communication interface 770, and/or the like) may determine relative locations associated with one or more ultrasound transmitters arranged to transmit ultrasound signals through a region of tissue during an ablation procedure and one or more ultrasound receivers arranged to receive the ultrasound signals transmitted by the one or more ultrasound transmitters after the ultrasound signals pass through the region of tissue, as described above.

As further shown in FIG. 15, process 1500 may include calculating a set of temperature-dependent measurements for the ultrasound signals transmitted through the region of tissue during the ablation procedure (block 1520). For example, the signal processor (e.g., using processor 720, memory 730, storage component 740, input component 750, output component 760, communication interface 770, and/or the like) may calculate a set of temperature-dependent measurements for the ultrasound signals transmitted through the region of tissue during the ablation procedure, as described above.

As further shown in FIG. 15, process 1500 may include determining, based on the set of temperature-dependent measurements and the relative locations associated with the one or more ultrasound transmitters and the one or more ultrasound receivers, one or more acoustic characteristics of the ultrasound signals transmitted through the region of tissue, wherein the one or more acoustic characteristics include one or more of a speed, an intensity, an attenuation, a phase, or a nonlinearity for the ultrasound signals (block 1530). For example, the signal processor (e.g., using processor 720, memory 730, storage component 740, input component 750, output component 760, communication interface 770, and/or the like) may determine, based on the set of temperature-dependent measurements and the relative locations associated with the one or more ultrasound transmitters and the one or more ultrasound receivers, one or more acoustic characteristics of the ultrasound signals transmitted through the region of tissue, as described above. In some implementations, the one or more acoustic characteristics include one or more of a speed, an intensity, an attenuation, a phase, or a nonlinearity for the ultrasound signals.

As further shown in FIG. 15, process 1500 may include generating an image representing a thermal map of the region of tissue during the ablation procedure based on temperature-dependent variations in the one or more acoustic characteristics of the ultrasound signals (block 1540). For example, the signal processor (e.g., using processor 720, memory 730, storage component 740, input component 750, output component 760, communication interface 770, and/or the like) may generate an image representing a thermal map of the region of tissue during the ablation procedure based on temperature-dependent variations in the one or more acoustic characteristics of the ultrasound signals, as described above.

Process 1500 may include additional implementations, such as any single implementation or any combination of implementations described below and/or in connection with one or more other processes described elsewhere herein.

In a first implementation, the relative locations associated with the one or more ultrasound transmitters and the one or more ultrasound receivers may be determined based on temperature-dependent measurements associated with ultrasound signals transmitted from the one or more ultrasound transmitters to the one or more ultrasound receivers before the ablation procedure.

In a second implementation, alone or in combination with the first implementation, the signal processor may obtain simulation data including an expected temperature evolution for the region of tissue during the ablation procedure based on a three-dimensional anatomical image of the region of tissue and one or more biophysical parameters. In some implementations, the image representing the thermal map of the region of tissue may be generated based on the expected temperature evolution for the region of tissue in combination with one or more tomographic image reconstruction techniques.

In a third implementation, alone or in combination with one or more of the first and second implementations, the signal processor may obtain simulation data including a simulated thermal map based on expected temperature-dependent measurements for the ultrasound signals to be transmitted through the region of tissue during the ablation procedure. In some implementations, the signal processor may perform an action based on a comparison of the set of temperature-dependent measurements for the ultrasound signals transmitted through the region of tissue during the ablation procedure and the expected temperature-dependent measurements for the ultrasound signals.

In a fourth implementation, alone or in combination with one or more of the first through third implementations, the image representing the thermal map of the region of tissue may be generated using one or more tomographic techniques based on the relative locations associated with the one or more ultrasound transmitters and the one or more ultrasound receivers.

Although FIG. 15 shows example blocks of process 1500, in some implementations, process 1500 may include additional blocks, fewer blocks, different blocks, or differently arranged blocks than those depicted in FIG. 15. Additionally, or alternatively, two or more of the blocks of process 1500 may be performed in parallel.

The foregoing disclosure provides illustration and description, but is not intended to be exhaustive or to limit the implementations to the precise forms disclosed. Modifications and variations may be made in light of the above disclosure or may be acquired from practice of the implementations.

As used herein, the term “component” is intended to be broadly construed as hardware, firmware, and/or a combination of hardware and software.

As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Certain user interfaces have been described herein and/or shown in the figures. A user interface may include a graphical user interface, a non-graphical user interface, a text-based user interface, and/or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, and/or the like). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods described herein may be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A system, comprising:
an ultrasound transmitter to transmit ultrasound signals through a region of tissue during an ablation procedure;
an ultrasound receiver to receive the ultrasound signals transmitted by the ultrasound transmitter after the ultrasound signals pass through the region of tissue; and
a signal processor, communicatively coupled to the ultrasound transmitter and the ultrasound receiver, to:
communicate with the ultrasound transmitter and the ultrasound receiver to obtain a set of measurements related to the ultrasound signals transmitted through the region of tissue during the ablation procedure;
determine one or more acoustic characteristics of the ultrasound signals transmitted through the region of tissue based on the set of measurements; and
generate an image representing a thermal map of the region of tissue during the ablation procedure based on a mapping between the one or more acoustic characteristics of the ultrasound signals and changes in temperature.

2. The system of claim 1, wherein the one or more acoustic characteristics include changes in one or more of speeds or intensities at which the ultrasound signals travel through the region of tissue, and
wherein the mapping used to generate the image representing the thermal map of the region of tissue is based on a relationship between changes in temperature and the changes in the speeds or intensities at which the ultrasound signals travel through the region of tissue.

3. The system of claim 2, wherein the mapping used to generate the image representing the thermal map of the region of tissue is further based on temperature-dependent variations in one or more of a time of flight, an attenuation, a phase, or a nonlinearity for at least one of the ultrasound signals transmitted through the region of tissue.

4. The system of claim 1, wherein:
the ultrasound receiver includes a transducer array having one or more transducer elements with known locations, and
the signal processor is further to:
determine a relative geometry between the ultrasound transmitter and the ultrasound receiver based on the known locations of the one or more transducer elements and time of flight data associated with ultrasound signals transmitted from the ultrasound transmitter to the ultrasound receiver before the ablation procedure.

5. The system of claim 4, wherein the signal processor is to generate the image representing the thermal map of the region of tissue using one or more tomographic techniques based on the relative geometry between the ultrasound transmitter and the ultrasound receiver.

6. The system of claim 1, wherein the signal processor, when generating the thermal map of the region of tissue, is further to:
use a thermal propagation model to segment the region of tissue into groups of voxels that have similar temperatures; and
reduce a region of interest to be represented by the thermal map to an area where the ultrasound signals are causing a change in temperature during the ablation procedure based on the groups of voxels that have the similar temperatures.

7. The system of claim 1, wherein the signal processor is further to:
obtain patient-specific simulation data including an expected temperature evolution for the region of tissue during the ablation procedure based on a three-dimensional anatomical image of the region of tissue and one or more biophysical parameters; and
use the patient-specific simulation data including the expected temperature evolution for the region of tissue in combination with one or more tomographic image reconstruction techniques to generate the image representing the thermal map of the region of tissue.

8. The system of claim 1, wherein the signal processor is further to:
obtain simulation data including a simulated thermal map based on expected time of flight measurements for the ultrasound signals to be transmitted through the region of tissue during the ablation procedure; and
perform an action based on a comparison of actual time of flight measurements for the ultrasound signals transmitted through the region of tissue during the ablation procedure and the expected time of flight measurements for the ultrasound signals.

9. A method, comprising:
obtaining, by a device, patient-specific simulation data including expected temperature-dependent measurements for ultrasound signals to be transmitted through a region of tissue during an ablation procedure;
determining, by the device, a relative geometry between an ultrasound transmitter arranged to transmit the ultrasound signals through the region of tissue during the ablation procedure and an ultrasound receiver arranged to receive the ultrasound signals transmitted by the ultrasound transmitter after the ultrasound signals pass through the region of tissue;
calculating, by the device, actual temperature-dependent measurements for the ultrasound signals transmitted through the region of tissue during the ablation procedure based on the relative geometry between the ultrasound transmitter and the ultrasound receiver; and
performing, by the device, an action to guide the ablation procedure based on a comparison of the actual temperature-dependent measurements for the ultrasound signals and the expected temperature-dependent measurements for the ultrasound signals.

10. The method of claim 9, wherein:
the patient-specific simulation data further includes a simulated thermal map based on the expected temperature-dependent measurements for the ultrasound signals, and
the action includes displaying the simulated thermal map to guide the ablation procedure based on the comparison indicating a threshold similarity between the actual temperature-dependent measurements and the expected temperature-dependent measurements for the ultrasound signals.

11. The method of claim 9, wherein the action includes causing the ablation procedure to stop based on the comparison indicating one or more of insufficient ablation in a targeted area of the region of tissue or off-target ablation in the region of tissue.

12. The method of claim 9, wherein:
the patient-specific simulation data further includes an expected temperature evolution for the region of tissue during the ablation procedure based on a three-dimensional anatomical image of the region of tissue and one or more biophysical parameters, and
the action includes using the expected temperature evolution for the region of tissue in combination with one or more tomographic image reconstruction techniques to generate an image representing a thermal map of the region of tissue during the ablation procedure.

13. The method of claim 12, wherein the expected temperature evolution for the region of tissue is represented according to one or more of a temporal resolution or a spatial resolution.

14. The method of claim 12, wherein the image is a synthesized thermal image generated using one or more of a deep learning technique or a machine learning technique.

15. The method of claim 12, wherein the image is an ultrasound elastography image based on pressure changes along the ultrasound signals transmitted through the region of tissue.

16. A non-transitory computer-readable medium storing instructions, the instructions comprising:
one or more instructions that, when executed by one or more processors, cause the one or more processors to:
determine relative locations associated with one or more ultrasound transmitters arranged to transmit ultrasound signals through a region of tissue during an ablation procedure and one or more ultrasound receivers arranged to receive the ultrasound signals transmitted by the one or more ultrasound transmitters after the ultrasound signals pass through the region of tissue;
calculate a set of temperature-dependent measurements for the ultrasound signals transmitted through the region of tissue during the ablation procedure;
determine, based on the set of temperature-dependent measurements and the relative locations associated with the one or more ultrasound transmitters and the one or more ultrasound receivers, one or more acoustic characteristics of the ultrasound signals transmitted through the region of tissue,
wherein the one or more acoustic characteristics include one or more of a speed, an intensity, an attenuation, a phase, or a nonlinearity for the ultrasound signals; and
generate an image representing a thermal map of the region of tissue during the ablation procedure based on temperature-dependent variations in the one or more acoustic characteristics of the ultrasound signals.

17. The non-transitory computer-readable medium of claim 16, wherein the relative locations associated with the one or more ultrasound transmitters and the one or more ultrasound receivers are determined based on temperature-dependent measurements associated with ultrasound signals transmitted from the one or more ultrasound transmitters to the one or more ultrasound receivers before the ablation procedure.

18. The non-transitory computer-readable medium of claim 16, wherein the one or more instructions further cause the one or more processors to:
obtain simulation data including an expected temperature evolution for the region of tissue during the ablation procedure based on a three-dimensional anatomical image of the region of tissue and one or more biophysical parameters,
wherein the image representing the thermal map of the region of tissue is generated based on the expected temperature evolution for the region of tissue in combination with one or more tomographic image reconstruction techniques.

19. The non-transitory computer-readable medium of claim 16, wherein the one or more instructions further cause the one or more processors to:
obtain simulation data including a simulated thermal map based on expected temperature-dependent measurements for the ultrasound signals to be transmitted through the region of tissue during the ablation procedure; and
perform an action based on a comparison of the set of temperature-dependent measurements for the ultrasound signals transmitted through the region of tissue during the ablation procedure and the expected temperature-dependent measurements for the ultrasound signals.

20. The non-transitory computer-readable medium of claim 16, wherein the image representing the thermal map of the region of tissue is generated using one or more tomographic techniques based on the relative locations associated with the one or more ultrasound transmitters and the one or more ultrasound receivers.
